(19) United States
(12) Patent Application Publication
(10) Pub. No.: US 2008/0249638 A1
(43) Pub. Date: Oct. 9, 2008

(54) BIODEGRADABLE THERAPEUTIC IMPLANT FOR BONE OR CARTILAGE REPAIR

(75) Inventor: Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(73) Assignee: CINVENTION AG, Wiesbaden (DE)

(21) Appl. No.: 12/098,722

(22) Filed: Apr. 7, 2008

Related U.S. Application Data
(60) Provisional application No. 60/910,454, filed on Apr. 5, 2007.

(51) Int. Cl. A61F 2/02; A61F 2/28

(52) U.S. Cl. 623/23.75; 623/16.11; 623/23.72

(57) ABSTRACT

The exemplary embodiments of the present invention relate to an at least partially biodegradable implant suitable for implantation into a subject for repairing a bone or cartilage defect, comprising a matrix forming an open-celled structure having a plurality of interconnected spaces, whereas the channels of the matrix are substantially completely filled with metallic material particles, and wherein at least one of the metallic material or the matrix material is at least partially degradable in-vivo. Furthermore, the present invention relates to a method for repairing a bone or cartilage defect in a living organism, comprising implanting an implant according to the exemplary embodiments of the present invention into the defective bone or cartilage, or replacing the defective bone or cartilage at least partially.

### CROSS-REFERENCE TO RELATED APPLICATION(S)

The present invention claims priority of U.S. provisional application Ser. No. 60/910,454 filed Apr. 5, 2007, the entire disclosure of which is incorporated herein by reference.

### FIELD OF THE PRESENT INVENTION

The present invention relates to an at least partially biodegradable implant suitable for implantation into a subject for repairing a bone or cartilage defect, comprising a matrix forming an open-celled structure having a plurality of interconnected spaces, wherein the channels of the matrix are substantially completely filled with metallic material particles, and wherein at least one of the metallic material or the matrix material is at least partially degradable in-vivo. Furthermore, the present invention, also relates to a method for repairing a bone or cartilage defect in a living organism, comprising implanting an implant according to the exemplary embodiments of the present invention into the defective bone or cartilage, or replacing the defective bone or cartilage at least partially.

### BACKGROUND INFORMATION

Implants are increasingly used in surgical, orthopedic, dental and other related applications, such as tissue engineering. However, the conventional implant technology is focused on improving implants by making them combination products, i.e., combining drugs or therapeutically active agents with implants, such as drug-eluting coatings, or by incorporating those agents into the implant body. Other research and development is generally focused on increasing the contact surface between the tissue and implant surface. In some specific treatment, bone defects are treated by using cements or cement-like materials comprising ceramic materials or polymer ceramic composites. In addition, the treatment of bone defects can involve the implantation of an autograft, an allograft, or a xenograft in the defected site. Biological implants and grafts suffer of many issues, such as shortage of donor tissue, infectious contamination by bacteria or virus and others. A synthetic implant may comprise in those cases potential alternatives.

An important issue is that due to biomechanical and physiologic requirements an implant material should have a certain mechanical strength or elasticity to be incorporated into the target tissue and anatomic region, on the other hand desired functions, such as degradability or incorporating beneficial agents, such as pharmacologically or therapeutically active agents are mostly contradictory the foregoing. For example, a range of bone grafting materials are established in clinical use, such as demineralized human bone matrix, bovine collagen mineral composites and processed coralline hydroxyapatite, calcium sulphate scaffolds, bioactive glass scaffolds and calcium phosphate scaffolds. Such orthopedic implants can be used as both temporary and permanent conduits for bone. Those materials may also be used to facilitate and direct the growth of bone or cartilage tissue across sites of fractures or to re-grow them in defective, damaged or infected bone. The provision of appropriate implants also requires considering the structure of bone that has to be treated. Cortical and cancellous bone are structurally different, although the material composition is very similar. Cancellous bone comprises a thin interstitium lattice interconnected by pores of 500-600 micron width with a spongy and open-spaced structure, whereby the interstitium can be substituted by a scaffolding material. Cortical bone comprises neurovascular “Haversian” canals of about 50-100 micron width within a hard or compact interstitium. A scaffold may allow at least osteoconduction or osteoinduction. Osteoinductive materials actively trigger and facilitate bone growth, for example by recruiting and promoting the differentiation of mesenchymal stem cells into osteoblasts. Osteoconductive materials induce bone to grow in areas where it would not normally grow, also called “ectopic” bone growth, usually by biochemical and/or physical processes. Osteogenic materials contain cells that can form bone or can differentiate into osteoblasts.

Particularly, it can be desirable to have an implant material that allows osseointegration. Known implants either provide a rough surface, usually made from metals, such as titanium, titanium alloys, stainless steel or cobalt chromium, or sometimes a porous surface. When using such materials, the osseointegration is typically only a mechanical integration that typically is poor or incomplete. Other reasons of incomplete integration are due to weak bone of the patient, for example due to cancerous diseases or osteoporosis. However, a rough or porous surface is usually applied to dense metal implants, for example by thermal spraying, surface abrasion, pitting, or other methods. Other solutions may provide a coating of hydroxyapatite, that usually is coated onto the surface of such conventional implants. It is a known issue that the adhesion of hydroxyapatite is not very strong and depending on the physiologic fluids present, in case of inflammation for example comprising acidic pH, the loosening of the hydroxyapatite occurs regularly.

Other reasons for implant failure are that dense implants are embedded non-physiologically into the surrounding tissue, inherently with suboptimal biomechanical integration into the part of the body or tissue, for example frequently causing micro fractures or, because of insufficient osseointegration, micro movements. One typical effect of implant failure, regardless of the real cause, is a peri-implantitis, acute, subacute or chronic inflammation that continuously affects or opposes the intended implant function. Specifically in critical implant regions, such as dental implants, the biologic environment and physiologic conditions is a complicating factor with a higher risk of infections due to the microbial, bacterial or fungi flora. Typical effects that may be caused by peri-implantitis are inflammation of mucosa, loss of attached gingival, exposure of a cervical portion of the implant and loss of the surrounding bone and functional implant failures. Even in dental treatments with extraction of a tooth an open wound is caused that might be contaminated by bacteria. A further significant issue is that the absence of the tooth induces spontaneously alveolar bone remodeling with resulting atrophy. Atrophy may subsequently cause more complex complications for reconstruction.

German Patent Publication DE 19901271 describes an implant for reconstruction of bone defects comprising a highly pure aluminum oxide and/or zirconium oxide ceramic, the surface of which is at least partially coated with tricalcium phosphate or hydroxylapatite. An independent claim is also included for a method of reconstructing bone defects by inserting the ceramic implant, where an implant (or a mold for casting an implant) corresponding to the image site is prepared using an imaging process and the implant is coated before insertion. U.S. Patent Publication No. 2005/249773 describes a degradable implant composition based on biocompatible ceramics and minerals, biocompatible glasses, and biocompatible polymers, and the use thereof for e.g., in-situ replicating a bone defect, or shaping an implant in a mold ex-situ. European Patent Publication No. 1344538 describes a method to produce and a porous biodegradable implant based on biocompatible ceramics, biocompatible glasses, biocompatible polymers, and combinations thereof. U.S. Pat. No. 5,282,861 describes a bone implant consisting of an open-celled tantalum structure formed by vacuum deposition of a thin tantalum layer onto a reticulated carbon foam, resulting in a lightweight porous structure mimicking the microstructure of cancellous bone for osteconduction. U.S. Pat. No. 6,087,553 describes an implant obtained by interdigitating polyethylene to a desired depth into the surface of an implant as described in U.S. Pat. No. 5,282,861 to provide a surface of the implant being smooth and having less friction. None of these documents teach or disclose filling the pore system of an open-celled matrix with degradable material particles.

There are several disadvantages related to the use of ceramic materials in implant materials. For example, the main disadvantage of using hydroxyl apatite crystalline forms in such materials is its lack of microporosity and mechanical stability. For adequate bone in-growth it is conventionally known that a porosity of, e.g., at least 100 μm or even more is required that cannot be obtained by ceramic or crystalline forms of hydroxyl apatite. Another drawback is the inferior mechanical stability of hydroxylapatite that is brittle and thus typically not suitable for stem replacement in implants. Conventional solutions with only coating a metal implant surface with hydroxyl apatite are prone to fatigue-related destruction of the coating. The application of hydroxyl apatite based cements further comprises a significant issue of mechanical stability and stress shielding as the formation of natural bone tissue is a physiologic process over time whereby during the engraftment phase the materials based on or including hydroxyl apatite do not provide a sufficient biomechanical stability unless the engraftment process is completed. The use of polymers also comprises constraints due to the fact that polymers are prone to suffer from creep and fatigue.

Metallic implant materials are usually favorable in terms of toughness, ductility and fatigue resistance. On the other hand they are known to be stiffer than natural bone, resulting in stress shielding. The phenomenon of stress shielding is well known and based on the effect that the implant material bears more of mechanical loads if it is stiffer than the surrounding tissue. This results in a “shielding” of the natural bone tissue from the mechanical load triggering the resorption processes of bone. Other ceramic implant materials are known to be prone to micro cracks, particularly when impulsive forces occur.

A further known issue is that several implant materials, particularly polymer or ceramic based materials are often hardly detectable by non-invasive imaging methods after implantation.

### SUMMARY OF EXEMPLARY EMBODIMENTS OF PRESENT INVENTION

One exemplary object of the present invention is to provide implants for orthopedic, surgical, dental and traumatologic implants, particularly implants for substituting or repairing e.g., bone defects.

For example, the implant can be made from materials that may provide an adjustable, accurate biodegradation in-vivo, and may be tailored to provide additional functions, such as incorporating or releasing beneficial agents.

According to an exemplary embodiment of the present invention, an at least partially biodegradable implant can be provided which is suitable for implantation into a subject for repairing a bone or cartilage defect. The exemplary implant comprises a matrix of a non-particulate material, the matrix forming an open-celled, three dimensional, lattice-like structure having a plurality of interconnected continuous spaces, and a plurality of particles of a metallic material, wherein the spaces, channels and/or pores of the matrix are substantially completely filled with the metallic material particles, and whereas at least one of the metallic material or the matrix material is at least partially degradable in-vivo. Typically, the pores or openings of the open-celled structure of the matrix material are substantially completely filled with the second material particles to provide a densely packed implant.

Such a structure can have osteoinductive or osteoconductive properties, e.g., it may actively trigger and facilitate bone growth, for example by recruiting and promoting the differentiation of mesenchymal stem cells into osteoblasts, it may induce bone to grow in areas where it would not normally grow, also called “ectopic” bone growth. For example, the matrix may have a bulk volume porosity of about 10-90%. The implant may form a structure, wherein the interconnected channels/pores define a spongy or trabecular open-spaced lattice structure of interconnecting spaces or channels within the matrix material. In exemplary embodiments, the channels/pores in the matrix material have a dimension e.g., diameter or length, suitable for osteoconduction, such as from about 200 to 1000 μm.

The exemplary implant may be used for repairing a bone, tooth or cartilage defect in a living organism by implanting the implant into a subject, such as a human being, in-vivo. Furthermore, the implant may be used to replace natural bone or cartilage in a living organism in-vivo. For example, the implant may be an implantable tissue replacement, an implantable fracture fixation device, such as plates, screws and rods, a dental implant, an orthopedic implant, a traumatologic implant, or a surgical implant.

In an exemplary embodiment of the present invention, the metallic material particles can include at least one of a metal or a metal alloy. Furthermore, the metallic material particles can be completely degradable in-vivo.

According to an alternative exemplary embodiment of the present invention, the metallic material particles are substantially not degradable in-vivo, but the matrix material is degradable, or both materials are degradable. In such embodiments, the in-vivo degradation rate of the matrix material and the metallic material particles are different to provide after implantation, the formation of an osteconductive, porous structure by preferential degradation of the faster degradable material. For example, in certain exemplary embodiments, the in-vivo degradation rate of the matrix material is lower than the degradation rate of the metallic material particles. In other exemplary embodiments, the in-vivo degradation rate of the matrix material is higher than the degradation rate of the metallic material particles, e.g., for dental applications.

In another exemplary embodiment, the metallic material particles are selected such that the in-vivo degradation rate of the particles substantially matches with the re-growth or repair rate of the natural bone, e.g., the degradation rate of the particles may be in a range of from about 3 to 8 weeks. In other exemplary embodiments, the metallic material particles are selected such that the in-vivo degradation rate of the particles substantially matches with the regrowth or repair rate of the natural cartilage, e.g., the degradation rate of the particles may be in a range of from about 4 to 10 weeks.

According to a further exemplary embodiment of the present invention, the implant can include particles of metallic material selected from a biocorrosive alloy, or a mixture of at least one first metallic material and at least one second metallic material, the first metallic material being more electronegative than the second metallic material, such that the first and second metallic material particles form a local cell at their contact surfaces. In such an exemplary embodiment, the less noble metal is preferentially degraded in-vivo.

In exemplary embodiments, the non-particulate matrix material includes an organic material, such as a polymer or copolymer, which may be a biodegradable polymer. In other exemplary embodiments, the matrix may itself consist of a metallic material, such as a metal or an alloy, or may consist of a ceramic material.

According to a further exemplary embodiment of the present invention, the matrix can include an inorganic-organic hybrid material, for example a material obtainable by sol-gel processing. Also, the matrix material may include a combination of any of the above described materials.

In addition, the implants of exemplary embodiments may further comprise known additives, such as a solvent, a filler, a pigment, or a beneficial agent, which may optionally be configured to be released in-vivo from the implant after insertion into the living organism.

According to a further exemplary embodiment of the present invention, a method for repairing a bone or cartilage defect in a living organism can be provided, comprising implanting an at least partially degradable implant as defined herein into the defective bone or cartilage, or replacing the defective bone or cartilage at least partially with the implant.

Another exemplary embodiment of the present invention is to provide a class of implants whereby the mechanical, chemical, biological and physical properties, such as electrical conductivity, optical or other suitable properties can be tailored appropriately to the intended use.

Another exemplary embodiment is that the scaffold or implant as described herein may comprise rationally designed structures to allow engraftment, ingrowth, induction or conduction or any combination thereof.

### BRIEF DESCRIPTION OF THE DRAWINGS

Further objects, features and advantages of the present invention will become apparent from the following detailed description taken in conjunction with the accompanying Figures showing illustrative embodiments of the present invention, in which:

FIG. 1 is a schematic illustration of an exemplary trabecular structure of a first material of the implant according to an exemplary embodiment of the present invention, e.g., which can mimic natural cancellous or “spongy” bone;

FIG. 2 is a schematic illustration of a part of an implant according to another exemplary embodiment of the present invention, having interconnected spaces/channels within an open-celled matrix, with the spaces being unfilled;

FIG. 3 is a schematic illustration of a part of an implant according to another exemplary embodiment of the present invention, having interconnected spaces/channels within the open-celled matrix, with the spaces being unfilled, e.g., with the first material not shown therein; and

FIG. 4 is a schematic diagram of a section of a part of an implant according to still another exemplary embodiment of the present invention, with the spaces being unfilled.

Throughout the Figures, the same reference numerals and characters, unless otherwise stated, are used to denote like features, elements, components or portions of the illustrated embodiments. Moreover, while the subject invention will now be described in detail with reference to the Figures, it is done so in connection with the illustrative embodiments. It is intended that changes and modifications can be made to the described embodiments without departing from the true scope and spirit of the subject invention.

### DETAILED DESCRIPTION OF EXEMPLARY EMBODIMENTS

The terms “active ingredient”, “active agent” or “beneficial agent” can include but not limited to any material or substance which may be used to add a function to the implantable medical device. Examples of such exemplary active ingredients can include biologically, therapeutically or pharmacologically active agents, such as drugs or medicaments, diagnostic agents, such as markers, or absorptive agents. The active ingredients may be a part of the first or second particles, such as incorporated into the implant or being coated on at least a part of the implant. Biologically or therapeutically active agents can comprise substances being capable of providing a direct or indirect therapeutic, physiologic and/or pharmacologic effect in a human or animal organism. A therapeutically active agent include but not limited to a drug, pro-drug or even a targeting group or a drug comprising a targeting group. An “active ingredient” according to an exemplary embodiment of the present invention may further include but not limited to a material or substance which may be activated physically, e.g., by radiation, or chemically, e.g., by metabolic processes.

The term “biodegradable” as used herein can include but not limited to any biocompatible material which can be removed in-vivo, e.g., by biocorrosion or biodegradation. Thus, any exemplary material, e.g., a metal or organic polymer that can be degraded, absorbed, metabolized, or which is resorbable in the human or animal body may be used either for a biodegradable metallic layer or as a biodegradable template in the exemplary embodiments of the present invention. In addition, as used in this description, the terms “biodegradable”, “bioabsorbable”, “resorbable”, and “biocorrodible” can encompass but not limited to materials that are broken down and may be gradually absorbed or eliminated by the body in-vivo, regardless whether these processes are due to hydrolysis, metabolic processes, bulk or surface erosion.

The term “non-particulate material” as used herein can possibly exclude materials having the form of a plurality of particles, thus, for example, the term can possibly exclude materials in the form of fibers, spheres, beads etc. as the matrix material.

The exemplary embodiment of the present invention is described in greater detail with reference to exemplary embodiments. The following description makes reference to numerous specific details in order to provide a thorough understanding of the present invention. However, each and every specific detail needs not to be employed to practice the present invention.

In exemplary embodiments, the present invention can provide a partially or completely degradable implant for healing of tissue defects, such as replacing or repairing bone or cartilage defects in a living organism in need thereof. The exemplary implant may also comprise an orthopedic fixation device, such as a rod, screw, nail or plate. Another exemplary embodiment includes to provide an implant in the form of a replica of the defective area, for direct replacement of the defective area, such as replacing bone defects induced by surgical craniotomy, or filling tooth roots for dental restoration.

With the implants of exemplary embodiments of the present invention, being partially or completely degradable in-vivo, an implant is provided, which after implantation can develop into a porous, trabecular structure, which can e.g., mimic the structure of cancellous or cortical bone, thus providing osteoconductive and/or osteoinductive properties. The implants of the present invention thus allow to replace natural bone or cartilage material with e.g., an essentially dense and mechanically resilient material directly after implantation. After a certain time in the body, at least a part of the implant is gradually degraded, for example the metallic particles filling the channels in the matrix, gradually leaving or releasing a porous, e.g., trabecular matrix structure which facilitates, guides or even promotes ingrowth of the natural tissue, thus leading to an “anchorage” of at least a part of the implant in the tissue into which it has been implanted. In case of a fully degradable implant, this will be gradually completely replaced over time by regrown natural tissue.

For example, suitable selection of the exemplary metallic material particles and/or the matrix material, wherein at least one of these materials is biodegradable, it is possible to provide an implant comprising a biocompatible material that exhibits the desired mechanical properties directly after implantation. Furthermore, it is possible to select the materials used and their combination and structural distribution in the implant such that due to an at least partial degradation of at least a part of the material, e.g., the metallic particles in the lattice structure of the matrix, whereby the degradation rate can be controlled, a porous structure is formed in the body which allows a stepwise ingrowth of surrounding tissue and an incorporation of the implant material over time, thus promoting healing of the wound or cavity filled. Thus, with the implants of certain exemplary embodiments of the present invention, a temporarily tailorable variation of the properties of the implant depending on the progress of healing of the defect may be provided.

Hence, before biodegradation starts, the implant allows to mechanically resist biomechanical loads while in the mid- and long-term at least a part of the implant will be replaced during degradation by ingrown tissue that increases the flexibility and biomechanical properties by substituted natural tissue. Another advantage is that the present invention, allows to additionally easily functionalize the implant, for example by incorporating functional compounds, such as radiopaque particles, such as biocompatible metals, or to tailor specifically the mechanical properties, such as flexibility by introducing e.g., fibers. Moreover, the incorporation of e.g., anti-microbial agents, such as silver or copper into the implant can allow to increase the anti-infective properties of the implant.

In an exemplary embodiment, the implant can be inserted into the defective area for replacement of bone or cartilage. For example, the presence of degradable metallic particles then leaves an open-celled, porous structure, comprising interconnected channels or pores in the matrix by degradation of the metal in-vivo. Vice versa, using a degradable matrix material will lead to a spongy, trabecular structure of the metallic particles left over after degradation of the matrix over time. Such structures may promote the growth of natural tissue, e.g., bone, so that the implant is step by step replaced by the normal, natural tissue. In certain exemplary embodiments, the implant may be designed from completely degradable materials, so that is completely vanishes from the body of the living organism after time, i.e. the implant fulfills only a temporary function.

According to an exemplary embodiment of the present invention, an implant suitable for implantation into a subject for repairing a bone or cartilage defect is provided, the implant comprising a matrix of a non-particulate material, the matrix forming an -celled lattice structure having a plurality of interconnected channels and/or pores, and a plurality of particles of a metallic material, wherein the channels/pores of the lattice structure are substantially completely filled with the metallic material particles, and wherein at least one of the metallic material or the matrix material is at least partially degradable in-vivo.

While in exemplary embodiments, the implant before implantation is preferably dense, and the open-celled structure is only developed/laid open by degradation of one of its constituents, e.g., the metallic particles, the implant and/or matrix may also have a porous structure, at least partially, before implantation, to facilitate access of physiologic fluids.

In certain exemplary embodiments, the matrix may form an open porous structure that has a bulk volume porosity of about 10-90%, preferably from about 30% to 80% and more preferably from 50% to 80%, and which is substantially completely filled with metallic material particles. The interconnected channels/pores may define a spongy or trabecular open-spaced lattice structure of interconnecting continuous channels within the matrix material, which allows tissue ingrowth after removal/degradation of the metallic particles. In an exemplary embodiment for bone repair, the channels/pores are macropores having a dimension suitable for osteoconduction, preferably of about 200 micrometer (μm) to 1000 μm. Pore sizes and porosities may be measured by adsorption methods conventionally used, e.g., N₂ or Hg-adsorption.

In an exemplary embodiment, at least one of the materials used, i.e. the metallic particles and/or the non-particulate matrix material is degradable in-vivo. For example, the matrix material may be substantially not degradable in-vivo, whereas the metallic material particles are degradable, or both the matrix material and the metallic material particles are degradable in-vivo. Preferably, the exemplary implant is adapted to provide, after degradation of first degradable materials, an open-celled, interconnected network of channels or pores or capillaries or combined compartments, whereby degradation can take place partially or completely in situ or in-vivo, i.e. in the living body. These compartments are delimited e.g., by the non-degradable or slower degradable second materials that demarcate the interconnected network of hollow channels or pores. In a further exemplary embodiment, the first degradable materials are the metallic material particles, and the second material comprises the matrix material.

When using degradable materials it can be desirable, that the degradation rate approximately matches to the re-growth or repair rate of the tissue treated. Typical biodegradation rates for maintaining the structure or structural integrity of a scaffold can be for example about 4-10 weeks for cartilage repair and about 3-8 weeks for bone repair. The mechanical requirements of the implants are highly dependant on the type of tissue being replaced, for example cortical bone has a Young Modulus of about 15-30 GPa, whereby cancellous (or spongy, trabecular) bone has a Young Modulus of about 0.01-2 GPa. Cartilage has a Young Modulus of less than about 0.001 GPa. It is desirable that the materials used for an implant in any particular case should reflect this as far as possible.

Thus, in an exemplary embodiment of the present invention, the combination of materials used for the implant is appropriately selected to provide an implant having a Youngs modulus corresponding to cancellous natural bone, preferably in the range from about 0.01 to about 2 GPa, preferably from about 0.1 to 1 GPa, more preferably from about 0.8 to 1 GPa.

In an other exemplary embodiment of the present invention, the combination of materials used for the implant is appropriately selected to provide an implant having a Youngs modulus corresponding to cortical natural bone, preferably in the range from about 15 to about 30 GPa, preferably from about 18 to 28 GPa, more preferably from about 22 to 27 GPa.

In an exemplary embodiment in which the matrix material and the metallic material particles are degradable in-vivo, the in-vivo degradation rate of the matrix material and the metallic material particles are different, e.g., the in-vivo degradation rate of the matrix material can be lower than the degradation rate of the metallic material particles or vice versa.

In exemplary embodiments, it can be preferred that the metallic material particles are selected such that the in-vivo degradation rate of the particles matches with the re-growth or repair rate of the natural bone, wherein the degradation rate of the particles is preferably in a range of from about 3 to 8 weeks, more preferably from 8 to 12 weeks and more preferably more than 3 months.

In other exemplary embodiments, the metallic material particles may be selected such that the in-vivo degradation rate of the particles matches with the regrowth or repair rate of the natural cartilage, whereas the degradation rate of the particles is preferably in a range of from about 4 to 10 weeks, more preferably from 8 to 12 weeks and more preferably more than 3 months.

Metallic Material Particles

In an exemplary embodiment of the present invention, the metallic material particles include at least one of a metal or a metal alloy, e.g., selected from main group metals of the periodic system, transition metals, such as copper, gold, silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum, or from rare earth metals, and alloys or any mixtures thereof.

The metallic particles used in some exemplary embodiments, are, without excluding others, e.g.—iron, cobalt, nickel, manganese or mixtures thereof, e.g., iron-platinum-mixtures, or as an example for magnetic metal oxides iron oxides and ferrites. Particularly for exemplary implants with magnetic or signaling properties in general, magnetic metals or alloys, such as ferrites, e.g., gamma-iron oxide, magnetite or ferrites of Co, Ni, Mn may be used. Examples are described in International Patent Publication Nos. WO83/03920, WO83/01738, WO85/02772, WO88/00060, WO89/03675, WO90/01295 and WO90/01899, and U.S. Pat. Nos. 4,452,773, 4,675,173 and 4,770,183. In certain exemplary embodiments, it is possible to select the particles from shape memory alloys, such as nickel titanium, nitinol, copper-zinc-aluminium, copper-aluminum-nickel, and the like.

In other exemplary embodiments, the particles are selected from biodegradable metals or alloys, metallic particle mixtures or metal composites. Suitable biodegradable metals can include, e.g., metals, or metal alloys, including alkaline or alkaline earth metals, Fe, Zn or Al, such as Mg, Fe or Zn, and optionally alloyed with or combined with other particles selected from Mn, Co, Ni, Cr, Cu, Cd, Pb, Sn, Th, Zr, Ag, Au, Pd, Pt, Si, Ca, Li, Al, Zn and/or Fe.

In addition, metal oxides, nitrides carbides, ceramic materials etc. may be added in certain exemplary embodiments, e.g., alkaline earth metal oxides or hydroxides, such as magnesium oxide, magnesium hydroxide, calcium oxide, and calcium hydroxide or mixtures thereof.

In further exemplary embodiments, the biodegradable metal particles may be selected from biodegradable or biocorrosive metals or alloys based on at least one of magnesium or zinc, or an alloy comprising at least one of Mg, Ca, Fe, Zn, Al, W, Ln, Si, or Y, such as e.g., a Mg—Ca alloy, Mg—Zn alloy, Mg—Al—Zn alloy, e.g., commercially available AZ91D, LAE442, AE21.

Furthermore, the metallic particles may be substantially completely or at least partially degradable in-vivo. Examples for suitable biodegradable alloys comprise e.g., magnesium alloys comprising more than about 90% of Mg, about 4-5% of Y, and about 1.5-4% of other rare earth metals, such as neodymium and optionally minor amounts of Zr, wherein the components are selected to add up to 100%.; or biocorrosive alloys comprising as a major component tungsten, rhenium, osmium or molybdenum, for example alloyed with cerium, an actinide, iron, tantalum, platinum, gold, gadolinium, yttrium or scandium.

In a further exemplary embodiment of the present invention, the degradable metallic material particles may comprise a metal alloy of (i) about 10-98 wt.-%, such as about 35-75 wt.-% of Mg, and about 0-70 wt.-%, such as about 30-40% of Li and about 0-12 wt.-% of other metals, or (ii) about 60-99 wt.-% of Fe, about 0.05-6 wt.-% Cr, about 0.05-7 wt.-% Ni and up to about 10 wt.-% of other metals; or (iii) about 60-96 wt.-% Fe, about 1-10 wt.-% Cr, about 0.05-3 wt.-% Ni and about 0-15 wt.-% of other metals, wherein the individual weight ranges are selected to add up to 100 wt.-% in total for each alloy.

For example, the metallic material includes one of Mg, Zn, Ca, whereby the metallic material forms upon its degradation in-vivo a substance that has osteoinductive properties.

In such embodiments, the metallic particles may be degraded to produce, e.g., hydroxyl apatite and hydrogen within the living body in the presence of physiologic fluids. Hydroxyl apatite may induce or guide ingrowths of natural surrounding tissue into the residual implant structure. This property of the exemplary implant material is especially advantageous for implants with a temporary function, but with sufficient mechanical stability compared to bioceramics or hydroxyl apatite or polymers alone.

For example, in a first exemplary step, a substantially dense implant is inserted, which is capable to immediately fulfill its functions, e.g., to provide mechanical support. Subsequently, during a period of several days, weeks or months, depending on the use of the implant, a part of the implant, e.g., the metallic particles, is degraded, leaving behind the open porous network structure of the matrix material.

Whereas the exemplary network structure may have an osteoconductive function during ingrowth of surrounding tissue, the degradation products of the metallic particles may additionally have osteoinductive properties, e.g., promoting the formation of new tissue. Overall, with the material composition and the structure of the implants of exemplary embodiments of the present invention, faster healing and/or better ingrowth of tissue or even complete replacement of the implant by natural tissue may be provided.

According to exemplary embodiments of the present invention, by alloying the aforesaid metals it is e.g., possible to tune the physiologic degradation rate from a few days up to 20 years. Moreover, by introducing precious metals either within the alloy, or as a part of the metallic particles in combination with less precious metal particles, or alternatively by applying a currency for example with an appropriate electrode or similar device, the degradation of the metallic particles can substantially be altered. Using an exemplary metal also allows to utilize the mechanical strength of these compounds and to realize tailored implants that both address the mechanical requirements e.g., immediately after implantation for supportive functions, as well as the biodegradability for later provision or facilitation of tissue ingrowth and incorporation of the residual implant material, if any, into the bone or other tissue.

For example, the exemplary implant composition of the exemplary embodiments of the present invention can rationally be tailored by suitably adjusting the metal composition to induce a controlled corrosion. Corrosion occurs when two metals, with different potentials, are in electrical contact while immersed or at least in contact in an electrically conducting corrosive liquid, such as physiologic fluids. Because the metals have different natural potentials in the liquid, a current will flow from the anodic (more electronegative) metal to the cathodic (more electropositive) metal, which will increase the corrosion of the anode. This additional corrosion is also called bimetallic corrosion. It is also referred to as a galvanic corrosion, dissimilar metal corrosion or contact corrosion. In general, the degradation reactions which occur are similar to those that would occur on a single, uncoupled metal, but the rate of attack is increased, sometimes dramatically. With some metal combinations the change in the electrode potential in the couple potential can induce corrosion which would not have occurred in the uncoupled state (e.g., pitting). The effect of coupling the two metals together can increase the corrosion rate of the anode and reduces or even suppresses corrosion of the cathode. Mostly, bimetallic corrosion occurs in solutions containing dissolved oxygen, and in most neutral and alkaline liquids the primary cathodic reaction is the reduction of dissolved oxygen, while in acidic liquids the cathodic reaction is often the reduction of hydrogen ions to hydrogen gas. Under uncoupled corrosion the anodic and cathodic reactions occur at small, local areas on the metal. In a bimetallic couple the cathodic reaction is more, or totally, on the electropositive member of the couple and the anodic reaction is mostly, or totally, on the electronegative component of the couple.

Using these exemplary principles, the corrosion applied to the metallic particles in the implants of embodiments of the present invention can be a rationally tailored corrosion that can be verified by selecting suitable metallic particles and/or combinations thereof with regard to their electronegativity or electropositivity.

According to the exemplary embodiments of the present invention, the particles may be selected from suitable shapes, such as tubes, fibers, fibrous materials or wires or spherical or dendritic or any regular or irregular particle form, and the preferred particle sizes are in, but not limited to, a range of about 1 nm (nanometer) up to 8000 μm (micrometer), preferably nano- or micro sized particles.

The exemplary metallic material particles useful according to the exemplary embodiments of the present invention can have an average (D50) particle size from about 0.5 nm to 5000 μm, preferably below about 1000 μm, such as from about 0.5 nm to 1,000 μm or below 500 nm, such as from about 0.5 nm to 500 nm, or from about 500 nm to 400 nm. Preferred D50 particle size distributions can be in a range of about 10 nm up to 1000 μm, such as between about 25 nm and 600 μm or even between about 30 nm and 250 μm. Particle sizes and particle distribution of nano-sized particles may be determined conventionally by spectroscopic methods, such as photo correlation spectroscopy, or by light scattering or laser diffraction techniques.

Concerning the corrosion control with regard to the metallic material particles, basically two approaches toward implant design may be used. The first exemplary approach is the combination of first metal or metal alloy particles with identical or similar electronegativity together with at least one second entity of metal or metal alloy particles with a different electronegativity that is sufficient to affect the corrosion rate of the first particles. The second basic exemplary approach is based on selecting particles that are alloyed, for example in nano-alloys, or core/shell particles or metal particles coated with a different metal that impacts the corrosion of one of its constituents. However, any combination of the foregoing approaches may also be used according to the exemplary embodiments of the present invention. For example, in one exemplary embodiment, magnesium particles are combined with Ag or Au particles whereby the presence of a non-precious and precious metal would result in a rapidly corrodible or erodible combination. In another exemplary embodiment, magnesium particles coated with magnesium oxide comprise a different corrosion rate compared to magnesium particles that are coated with silver oxide.

According to another exemplary embodiment of the present invention, the metallic material particles comprises a mixture of at least one first metallic material and at least one second metallic material, the first metallic material being more electronegative than the second metallic material, such that the first and second metallic material particles form a local cell at their contact surfaces. In such an exemplary embodiment, the less noble metal is preferentially degraded in-vivo.

In another exemplary embodiment of the present invention, the size and surface-to-volume ratio of the metallic particles may be used to control the corrosion rate. For example, when using the same degradable metal or metal alloy particle but in different sizes, the smaller particles or those with a higher surface-to-volume ratio are typically prone to a higher corrosion rate. Therefore, even using the same metallic basically still allows to tailor the corrosion rate by selecting the appropriate particle size or combination of particle sizes. However, also a combination of different material composition as well as different particles comprising significantly different surface-to-volume ratios can be combined. Preferably, the exemplary composition used comprises particles including metals with different electronegativities to tailor the basic corrosion rate of the implant with an appropriate alloy.

In further exemplary embodiments, it can be preferred to have a rationally designed distribution of the metallic material particles and the matrix material within the implant body. Such a distribution may e.g., be influenced by selecting appropriate amounts and sizes of the materials used.

The exemplary metallic particles as described above are incorporated in the implants of the present invention within a non-particulate matrix material.

Exemplary Non-Particulate Matrix Material

According to an exemplary embodiment, the implant as described herein can include an organic material as the non-particulate matrix material.

For example, the organic material may comprise an oligomer, polymer or copolymer, such as a poly(meth)acrylate, unsaturated polyester, saturated polyester, polyolefines, polyethylene, polypropylene, polybutylene, alkyd resins, epoxy-polymers or resins, polyamide, polyimide, polyetherimide, polyamideimide, polyesterimide, polyester amide imide, polyurethane, polycarboxylate, polycarbonate, polystyrene, polyphenol, polyvinyl ester, polysilicone, polyacetal, cellulosic acetate, polyvinylchloride, polyvinyl acetate, polyvinyl alcohol, polysulfone, polyphenylsulfone, polyethersulfone, polyketone, polyetherketone, polybenzimidazole, polybenzoxazole, polybenzthiazole, polyfluorocarbons, polyphenylene ether, polyarylate, or cyanatoester-polymers, and any of the copolymers and any mixtures thereof.

One exemplary option is to use a biocompatible, but non-degradable polymer, such as polymethylmethacrylate and/or other acrylic co-polymers, preferably acrylic-terminated butadiene-styrene block copolymers, or cyanoacrylates, polyetherketone or polyetheretherketone, pre-polymers or any mixture thereof. Alternatively, a biodegradable polymer may be used.

According to an exemplary embodiment of the present invention, the organic material comprises a biocompatible and/or biodegradable polymer or copolymer, such as collagen, albumin, gelatin, hyaluronic acid, starch, cellulose, methylcellulose, hydroxypropylcellulose, hydroxypropylmethyl-cellulose, carboxymethylcellulose-phtalate; gelatine, casein, dextrane, polysaccharide, fibrinogen, poly(D,L lactide), poly(D,L-lactide-co-glycolide), poly(glycolide-co-trimethylene carbonates), poly(glycolide), poly(hydroxybutylate), poly(alkylcarbonate), poly(a-hydroxyesters), poly(ether esters), poly(orthoester), polyester, poly(hydroxyvaleric acid), polydioxanone, poly(ethylene terephtalate), poly(maleic acid), poly(malic acid), poly(tartaric acid), polyanhydride, polyphosphazene, poly(amino acids), polypeptides, polycaprolactones, poly(propylene fumarates), poly(ester amides), poly(ethylene fumarates), poly(hydroxy butyrates), and polyurethanes, or mixtures thereof. In such embodiments, the organic material may be selected from partially or substantially completely biodegradable polymers.

Further polymers which may be used include, for example, poly(meth)acrylate, unsaturated polyester, saturated polyester, polyolefines, such as polyethylene, polypropylene, polybutylene, alkyd resins, epoxy-polymers or resins, polyamide, polyimide, polyetherimide, polyamideimide, polyesterimide, polyester amide imide, polyurethane, polycarbonate, polystyrene, polyphenol, polyvinyl ester, polysilicone, polyacetal, cellulosic acetate, polyvinylchloride, polyvinyl acetate, polyvinyl alcohol, polysulfone, polyphenylsulfone, polyethersulfone, polyketone, polyetherketone, polybenzimidazole, polybenzoxazole, polybenzthiazole, polyfluorocarbons, polyphenylene ether, polyarylate, cyanatoester-polymers, and mixtures or copolymers of any of the foregoing.

In certain exemplary embodiments, the polymer material can be selected from poly(meth)acrylates based on mono(meth)acrylate, di(meth)acrylate, tri(meth)acrylate, tetra-acrylate and penta-acrylate monomers; as well as mixtures, copolymers and combinations of any of the foregoing, wherein the metallic particles may be included already during polymerization.

For example, the matrix may be a polymerization product of a monofunctional monomer, such as at least one of methyl acrylate, methyl methacrylate, ethyl acrylate, ethyl methacrylate, butyl acrylate, butyl methacrylate, acryl acrylate, acryl methacrylate, hydroxyethyl acrylate, hydroxyethyl methacrylate, methoxyethyl acrylate, and methoxyethyl methacrylate; or a polymerization product of a polyfunctional monomer which may include at least one of bifunctional aliphatic acrylates, bifunctional aliphatic methacrylates, bifunctional aromatic acrylates, bifunctional aromatic methacrylates, trifunctional aliphatic acrylates, trifunctional aliphatic methacrylates, tetrafunctional acrylates, and tetrafunctional methacrylates, such as triethylene glycol diacrylate, triethylene glycol dimethacrylate, 2,2-bis(4-methacryloxyphenyl)propane, 2,2-bis(4-methacryloxyethoxyphenyl)propane, 2,2-bis(4-methacryloxypolyethoxyphenyl)propane, 2,2-bis[4-(3-methacryloxy-2-hydroxypropoxy)-phenyl]propane, di(methacryloxyethyl) trimethylhexamethylene diurethane, tetramethylolmethane tetraacrylate, and tetramethylolmethane tetramethacrylate, or a di(meth)acrylate, such as urethane dimethacrylate, ethyleneglycol dimethacrylate, (2,2-bis[4-(2-hydroxy-3-methacryloxypropoxy)phenyl]propane (BIS-GMA), (2,2-bis[4-(methacryloxy)phenyl]propane (BIS-MA), 2,2-bis(4-methacryloxyphenyl)propane, 1,2-butanediol-diacrylate, 1,4-butanediol-diacrylate, 1,4-butanediol-dimethacrylate, 1,4-cyclo-hexanediol-dimethacrylate, 1,10-decanediol-dimethacrylate, diethylene-glycol-diacrylate, dipropyleneglycol-diacrylate, dimethylpropanediol-dimethacrylate, triethyleneglycol-dimethacrylate (TEGDMA), tetraethyleneglycol-dimethacrylate, 1,6-hexanediol-diacrylate, 1,6-bis-[2-methacryloxyethoxycarbonylamino]-2,2,4-trimethylhexane (UDMA), neopentylglycol-diacrylate, polyethyleneglycol-dimethacrylate, tripropyleneglycol-diacrylate, 2,2-bis-[4-(2-acryloxyethoxy)phenyl]-propane, 2,2-bis-[4-(2-hydroxy-3-methacryloxypropoxy)phenyl]propane, bis(2-methacryloxyethyl)N,N-1,9-nonylene-biscarbamate, 1,4-cyclohexanedimethanol-dimethacrylate, and diacrylic urethane oligomers. Also, mixtures of monofunctional and polyfunctional monomers may be used.

Exemplary Sol-Gel-Systems

According to a further exemplary embodiment of the present invention, the matrix material can include an inorganic-organic hybrid material, for example a material obtainable by conventional sol-gel processing or combined sol-gel-processing and polymerization reactions. The exemplary sol-gel processing can be either a hydrolytic or non-hydrolytic sol-gel processing, for example by using sol-gel forming materials including at least one metal alkoxide.

For example, the metal alkoxide can be selected from at least one of silicon alkoxides, tetraalkoxysilanes, oligomeric forms of tetraalkoxysilanes, alkylalkoxysilanes, aryltrialkoxysilanes, (meth)acrylsilanes, phenylsilanes, oligomeric silanes, polymeric silanes, epoxysilanes; fluoroalkylsilanes, fluoroalkyltrimethoxysilanes, or fluoroalkyltriethoxysilanes, optionally further comprising at least one crosslinking agent including at least one of isocyanates, silanes, (meth)acrylates, 2-hydroxyethyl methacrylate, propyltrimethoxysilane, 3-(trimethylsilyl)propyl methacrylate, isophoron diisocyanate, hexamethylenediisocyanate (HMDI), diethylenetriaminoisocyanate, 1,6-diisocyanatohexane, or glycerin.

In a further exemplary embodiment, the matrix may be obtained from a reaction mixture comprising a metal alkoxide including a hydrolytically condensable, organically modified trialkoxysilane which contains free-radically polymerizable acrylate or methacrylate groups or cyclic groups capable of ring opening polymerization. Examples include, e.g., those based on polysilicid acid modified with polymerizable alkoxy groups or cyclic siloxanes and a mixture of Bis-GMA and 2-hydroxyethyl methacrylate (HEMA). These materials can be cured by hydrolysis and condensation with simultaneous radical polymerization of the resultant alcohols. Functionalized trialkoxysilanes of the R—Si(OR′)₃ type may also be used (with e.g., R and/or R′ representing C₁ to C₂₀ alkyl, alkenyl or alkinyl, wherein R can include at least one acrylic or methacrylic acid functionality), which can condensate, resulting in polysilsesquioxanes RSiO_{3/2}, or which can be co-condensated with other alkoxysilanes or metal alkoxides. Methacrylates may also be used in combination with e.g., tetraethylorthosilicate (TEOS) to provide PMMA-silica hybrides as the matrix material after curing by polymerization and co-condensation.

An overview on several of these precursors for inorganic-organic hybrid materials suitable for the matrix material of the present invention is disclosed in N. Moszner and S. Klapdohr, Nanotechnology for dental composites, Int.J. of Nanotechnology, vol. 1, No. ½, 2004, 130-156, and the references cited therein. All materials referred to and mentioned therein are in principle also suitable for producing the matrix material in the implants of the present invention. For example, hydrolysable and condensable trialkoxysilanes bearing methacrylate groups can be used, which are connected to the Si-atom via spacers, and silanediacrylates can be preferred materials which can be hydrolysed and condensated into fluid sols, and cured by e.g., visible light by polymerization of the methacrylate functions.

The exemplary precursor compounds of an inorganic-organic hybrid material processible by sol-gel processing may be conventional sol/gel-forming components. The sol/gel-forming components are typically provided in the form of a sol which may comprise a solvent, and which can be cured or hardened by condensation into a gel, such as an aerogel or xerogel.

In these exemplary embodiments, degradable and non degradable metallic particles selected as described above can be combined and mixed with the sol/gel-forming components, or specifically only degradable or non-degradable particles can be used. Optionally, the gel obtained after curing is dissolvable in physiologic fluids, or porous.

In certain exemplary embodiments, the sol/gel forming components can include metal oxides, metal carbides, metal nitrides, metaloxynitrides, metalcarbonitrides, metaloxycarbides, metaloxynitrides, and metaloxycarbonitrides of the above mentioned metals, or any combinations thereof. These compounds, preferably as colloidal particles, can be reacted with oxygen-containing compounds, e.g., alkoxides to form a sol/gel.

If the matrix comprises a material obtainable by sol-gel processing, substantially all materials and processes as described in International Patent Publication No. WO 2006/077256 may be used.

In an exemplary embodiment, the matrix material may include a combination of any of the above described embodiments of the present invention. For example, hydrolytically condensable metal alkoxides used in sol-gel processing may include at least one polymerizable monofunctional or polyfunctional organic residue, which can be additionally or subsequently subjected to polymerization to produce the matrix material, and such materials may be combined with polymers or the like.

In addition, the implants of exemplary embodiments may further comprise conventional additives, such as a filler, e.g., salts, hydroxyl apatite; a pigment, or a beneficial agent as further described herein below, which may optionally be configured to be released in-vivo from the final implant

According to an exemplary embodiment of the present invention, the particles of metallic material comprise at least about 5 wt.-%, preferably from about 1 to 99 wt.-%, more preferably about 10 to 80 wt.-%, more preferably about 40 to 75 wt-% of the implants constituents. Furthermore, the metallic material particles can be modified with a coupling agent, preferably a silane coupling agent, such as vinyl trichlorosilane, vinyl triethoxysilane, vinyl trimethoxysilane, vinyl tris(beta-methoxyethoxy)silane, and gamma-methacryloxypropyl trimethoxysilane, to improve adherence in the matrix material or to covalently bond the particles to the matrix material.

In other exemplary embodiments, the matrix may itself consist of a metallic material, such as a metal or an alloy, or may consist of a ceramic material. Suitable such materials include all biocompatible metals and alloys as well as ceramic materials, including those as described above as materials for the metallic material particles.

According to further exemplary embodiments of the present invention, the implant after implantation facilitates and enables the formation and organization of tissue, preferably osteoinduction, osteoconduction and formation of natural bone minerals “guided” by the implant fine-structure.

Exemplary Manufacturing

The exemplary manufacture of the implant may be done by any suitable conventional manufacturing method. Appropriate exemplary techniques include molding a suitable precursor composition in a mold or replica form of the defect to be repaired with the desired design. Also, for example an injection molding processes can be applied. Other exemplary methods include compression molding, compacting, dry pressing, cold isostatic pressing, hot pressing, uniaxial or biaxial pressing, extrusion molding, gel casting, slip casting and tape casting and the like.

The exemplary implant must not be necessarily porous before implantation or use. It can be made of densely welded parts. The metallic material may also comprise welded or sintered particles, such as sintered pearls, selected and combined as described before, forming a 3-dimensional network structure embedded in a matrix.

In certain exemplary embodiments, it is possible to have a rationally designed distribution of the metallic material particles within the implant body, e.g., in the form of a trabecular, spongy structure capable to guide tissue growth along pathways released over time by degradation of the matrix material.

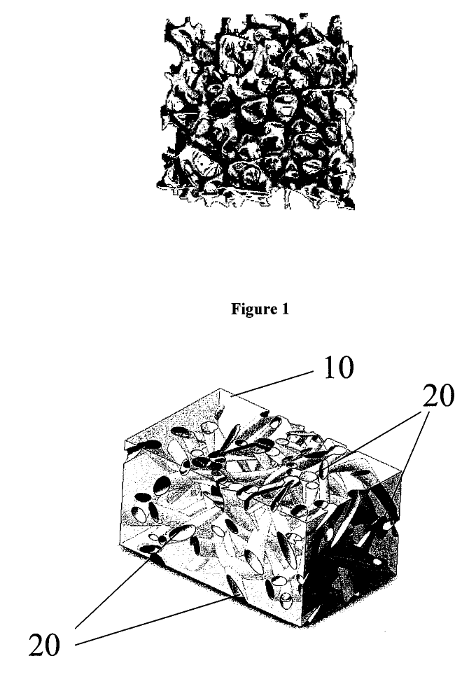

For example, FIG. 1 shows an exemplary trabecular, spongy structure of a part of an implant according to the exemplary embodiments of the present invention, similar to natural cancellous bone. In one exemplary embodiment, the structure shown in FIG. 1 represents an aggregation of metallic material particles embedded in a non-particulate matrix material (not shown).

According to another exemplary embodiment, the structure shown in FIG. 1 may represent a non-particulate matrix material in the from of a network structure, wherein the open space represents the network of an interconnected space to be filled with metallic material particles (not shown), i.e. the inverse of the above embodiment.

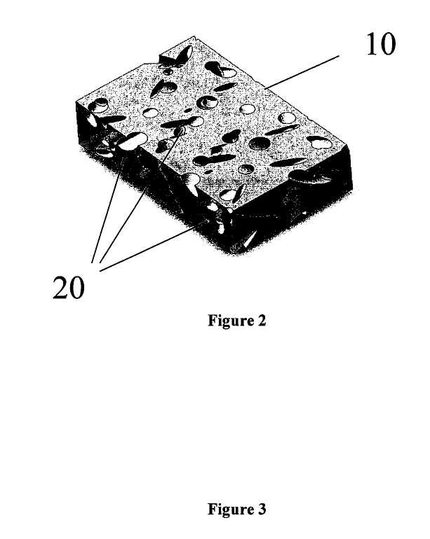

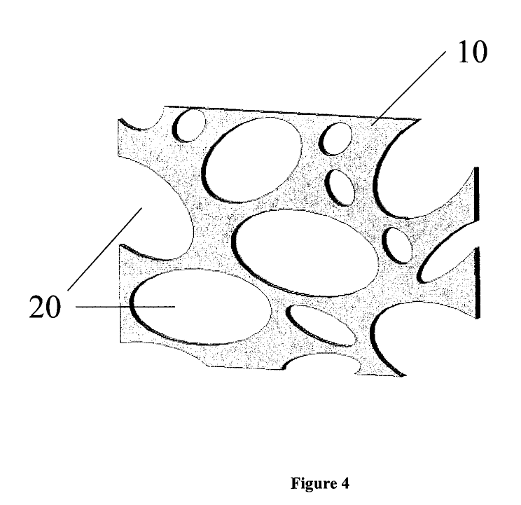

Alternative embodiments of the implant structure of the present invention, are shown in FIGS. 2 to 4. An open-celled matrix 10 is shown, having a plurality of interconnected spaces or channels 20 extending from the surface of the matrix through its interior, forming a network structure or framework of channels 20. After filling the space/channels 20 with the particulate metallic material, a substantially dense implant structure is obtained, which after implantation and degradation of e.g., the particulate metallic material provides a hollow structure in the matrix which guides the ingrowth of surrounding natural tissue.

For example, the matrix may also have a structure and may be prepared as described in U.S. Pat. No. 5,282,861, e.g., an open porous polymeric foam or a material derived therefrom, the pores or spaces thereof being filled with metallic material particles as described herein, wherein at least one of the matrix or the particles is biodegradable.

Exemplary manufacturing can be done by various conventional methods. The exemplary implants can be manufactured in one seamless part or with seams out of multiple parts. The present invention, also contemplates the use of different materials for different sections or parts of the exemplary implant. The exemplary implants may be manufactured using conventional implant manufacturing techniques. Particularly, appropriate manufacturing methods can include, but are not limited to, laser cutting, chemical etching or stamping of tubes. Another option is the manufacturing by laser cutting, chemically etching, and stamping flat sheets, rolling of the sheets and, as a further option, welding the sheets. Other appropriate manufacturing techniques include electrode discharge machining or molding the exemplary implant with the desired design. A further option is to weld or glue individual sections together. Any other suitable implant manufacturing process may also be applied and used. For example, for degradably alloyed implants conventional welding methods are appropriate, or it is possible to structure them, for example introducing open-cellular pores, by foaming or similar methods. Other suitable exemplary methods can include compression molding, compacting, dry pressing, cold isostatic pressing, hot pressing, uniaxial or biaxial pressing, extrusion molding, gel casting, slip casting and tape casting and the like. An exemplary method may be coextruding the metallic particles with organic matrix materials, or preparing an open-celled matrix by foaming and subsequently filling the channels/pores with metallic particles.

For example, in a further typical procedure, the open-celled framework is made from a metallic material by conventional methods as described above, such as manufacturing of porous metal implants by sintering of green bodies, bonding of metal sheets that are perforated by direct laser machining, abrasive water jet machining, stamping (e.g., computer numerical controlled (CNC) stamping), drilling, punching, ion beam or electrochemical or photochemical etching, electrical discharge machining (EDM), or other perforation techniques and/or combinations thereof. The open-celled matrix can then be filled with a particulate material by conventional methods such as, for example, depending on the dimensions of the open-celled framework structure and the size of particles, spraying, dipping, powder spraying, vacuum powder infiltration/impregnation, or, if the matrix is polymeric, polymerizing the particles into the matrix, particularly by adding the particles during foaming of the polymeric materials, etc., to obtain a substantially densely packed implant, wherein the pores in the first material are substantially completely filled with the second material.

The basic exemplary design of the implants of the exemplary embodiments of the present invention contemplates, that degradation and preferably formation of degradation products such as hydroxyl apatite or the in-growth and engraftment is “guided” as aforesaid. In exemplary embodiments, the implant may be shaped as desired, in the form of tubes or sheets or foils or meshes or the like, and then manufactured or welded to the final implant material and/or implant design. Preferably, the parts used comprise different metals, metal oxides or metal alloys. In one exemplary embodiment sheets of matrix material are cut to comprise a porous pattern, mesh-like pattern, trabecular pattern, random or pseudo-random structure or any mixture thereof. They can be stacked together in a sandwich-like manner to provide a three dimensional interconnected network of channels, pore or capillaries or combined compartments, serving as the matrix, which is then filled with the particles. Those sheets can be processed to different geometric forms, but however, the sheets can be welded or bonded together to a compact material, for example layer by layer. Preferably, those sheets or foils provide a degradable material themselves, but in certain exemplary embodiments, it can be preferred to use different materials in different layers to control corrosion and degradation of specific structural parts of the implant. For example, in certain exemplary embodiments, it can be preferred to have alternating layers of a degradable metal or metal alloy and non-degradable metal or metal alloys, if the matrix material is a metallic material itself. In other certain exemplary embodiments, it is possible to have alternating layers of a faster degradable material, e.g., a metal alloy or polymer and slower degradable materials, e.g., metal alloys or polymers.

In further exemplary embodiments, the pre-formed open-celled structure is manufactured as the ex-situ form previously, such as described in U.S. Pat. No. 5,282,861, before filling with the particles. The channels or pores are then filled up with single or mixed entities of the metallic material particles. Additionally, other particles of metals, metal oxides, metal alloys, ceramics, organics, polymers or composites or any mixture thereof, may simultaneously be added during filling of the channels/pores.

The basic design of the implants of the exemplary embodiments of the present invention contemplates, that degradation and preferably formation of degradation products, such as hydroxyl apatite or the in-growth and engraftment is “guided” as indicated herein.

The exemplary embodiments can comprise both an open-celled lattice structure as a degradable matrix structure as well as a non-degradable matrix in any desired three-dimensional orientation or shape.

Exemplary Functionalization

According to the exemplary embodiments of the present invention, additional functions may be provided in the implant by incorporating beneficial agents into at least a part of the implant structure, as desired. Beneficial agents can be selected from biologically active agents, pharmacological active agents, therapeutically active agents, diagnostic agents or absorptive agents or any mixture thereof. Furthermore, the implant may optionally be coated with beneficial agents partially or completely.

Biologically, therapeutically or pharmaceutically active agents according to the exemplary embodiments of the present invention may include a drug, pro-drug or even a targeting group or a drug comprising a targeting group. The active agents may be in crystalline, polymorphous or amorphous form or any combination thereof in order to be used in the present invention. Suitable therapeutically active agents may be selected from the group of enzyme inhibitors, hormones, cytokines, growth factors, receptor ligands, antibodies, antigens, ion binding agents, such as crown ethers and chelating compounds, substantial complementary nucleic acids, nucleic acid binding proteins including transcriptions factors, toxines and the like. Examples of active agents are, for example, cytokines, such as erythropoietine (EPO), thrombopoietine (TPO), interleukines (including IL-1 to IL-17), insulin, insulin-like growth factors (including IGF-1 and IGF-2), epidermal growth factor (EGF), transforming growth factors (including TGF-alpha and TGF-beta), human growth hormone, transferrine, low density lipoproteins, high density lipoproteins, leptine, VEGF, PDGF, ciliary neurotrophic factor, prolactine, adrenocorticotropic hormone (ACTH), calcitonin, human chorionic gonadotropin, cortisol, estradiol, follicle stimulating hormone (FSH), thyroid-stimulating hormone (TSH), leutinizing hormone (LH), progesterone, testosterone, toxines including ricine and further active agents, such as those included in Physician's Desk Reference, 58th Edition, Medical Economics Data Production Company, Montvale, N.J., 2004 and the Merck Index, 13th Edition (particularly pages Ther-1 to Ther-29), all of which are incorporated herein by reference.

In a further exemplary embodiment, the therapeutically active agent is selected from the group of drugs for the therapy of oncological diseases and cellular or tissue alterations. Suitable therapeutic agents are, e.g., antineoplastic agents, including alkylating agents, such as alkyl sulfonates, e.g., busulfan, improsulfan, piposulfane, aziridines, such as benzodepa, carboquone, meturedepa, uredepa; ethyleneimine and methylmelamines, such as altretamine, triethylene melamine, triethylene phosphoramide, triethylene thiophosphoramide, trimethylolmelamine; so-called nitrogen mustards, such as chlorambucil, chlornaphazine, cyclophosphamide, estramustine, ifosfamide, mechlorethamine, mechlorethaminoxide hydrochloride, melphalan, novembichin, phenesterine, prednimustine, trofosfamide, uracil mustard; nitroso urea-compounds, such as carmustine, chlorozotocin, fotenmustine, lomustine, nimustine, ranimustine; dacarbazine, mannomustine, mitobranitol, mitolactol; pipobroman; doxorubicin and cis-platinum and its derivatives, and the like, combinations and/or derivatives of any of those described herein.

In a further exemplary embodiment, the therapeutically active agent is selected from the group of anti-viral and anti-bacterial agents, such as aclacinomycin, actinomycin, anthramycin, azaserine, bleomycin, cuctinomycin, carubicin, carzinophilin, chromomycines, ductinomycin, daunorubicin, 6-diazo-5-oxn-1-norieucin, doxorubicin, epirubicin, mitomycins, mycophenolsäure, mogalumycin, olivomycin, peplomycin, plicamycin, porfiromycin, puromycin, streptonigrin, streptozocin, tubercidin, ubenimex, zinostatin, zorubicin, aminoglycosides or polyenes or macrolid-antibiotics, and the like, combinations and/or derivatives of any of those described herein.

In yet another exemplary embodiment, the therapeutically active agent is selected from the group of radio-sensitizer drugs.

In still another exemplary embodiment, the therapeutically active agent is selected from the group of steroidal or non-steroidal anti-inflammatory drugs.

In an additional exemplary embodiment, the therapeutically active agent is selected from agents referring to angiogenesis, such as, e.g., endostatin, angiostatin, interferones, platelet factor 4 (PF4), thrombospondin, transforming growth factor beta, tissue inhibitors of the metalloproteinases-1, -2 and -3 (TIMP-1, -2 and -3), TNP-470, marimastat, neovastat, BMS-275291, COL-3, AG3340, thalidomide, squalamine, combrestastatin, SU5416, SU6668, IFN-[alpha], EMD121974, CAI, IL-12 and IM862 and the like, combinations and/or derivatives of any of the foregoing.

In a still further exemplary embodiment, the therapeutically-active agent is selected from the group of nucleic acids, wherein the term nucleic acids also comprises oliogonucleotides wherein at least two nucleotides are covalently linked to each other, for example in order to provide gene therapeutic or antisense effects. Nucleic acids preferably comprise phosphodiester bonds, which also comprise those which are analogues having different backbones. Analogues may also contain backbones, such as, for example, phosphoramide (Beaucage et al., Tetrahedron 49(10):1925 (1993) and the references cited therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)); phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidit-compounds (see Eckstein, Oligonucleotides and Analogues: A Practical Approach, Oxford University Press), and peptide-nucleic acid-backbones and their compounds (see Egholm, J. Am. Chem. Soc. 114:1895 (1992); Meier et al., Chem. Int. Ed. Engl: 31:1008 (1992); Nielsen, Nature, 365:566 (1993); Carlsson et al., Nature 380:207 (1996), whereas these references are incorporated by reference herein in their entireties. Further analogues can include those having ionic backbones, see Denpcy et al., Proc. Natl. Acad. Sci. USA 92:6097 (1995), or non-ionic backbones, see U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996), and non-ribose-backbones, including those which are described in U.S. Pat. Nos. 5,235,033 and 5,034,506, and in chapters 6 and 7 of ASC Symposium Series 580, “Carbohydrate Modifications in Antisense Research”, Ed. Y. S. Sanghui and P. Dan Cook. The nucleic acids having one or more carbocylic sugars are also suitable as nucleic acids for use in the present invention, see Jenkins et al., Chemical Society Review (1995), pages 169 to 176 as well as others which are described in Rawls, C & E News, 2 Jun. 1997, page 36, herewith incorporated by reference. Besides the selection of the nucleic acids and nucleic acid analogues known in the conventional, also any mixtures of naturally occurring nucleic acids and nucleic acid analogues or mixtures of nucleic acid analogues may be used.

In a further exemplary embodiment, the therapeutically active agent is selected from the group of metal ion complexes, as described in International Applications PCT/US95/16377, PCT/US95/16377, PCT/US96/19900, PCT/US96/15527 and herewith incorporated by reference, wherein such agents reduce or inactivate the bioactivity of their target molecules, preferably proteins, such as enzymes.

Preferred therapeutically active agents are also anti-migratory, anti-proliferative or immune-supressive, anti-inflammatory or re-endotheliating agents, such as, e.g., everolimus, tacrolimus, sirolimus, mycofenolate-mofetil, rapamycin, paclitaxel, actinomycine D, angiopeptin, batimastate, estradiol, VEGF, statines and others, their derivatives and analogues.

Further exemplary can be active agents or combinations of active agents selected from heparin, synthetic heparin analogs (e.g., fondaparinux), hirudin, antithrombin III, drotrecogin alpha; fibrinolytics, such as alteplase, plasmin, lysokinases, factor XIIa, prourokinase, urokinase, anistreplase, streptokinase; platelet aggregation inhibitors, such as acetylsalicylic acid [aspirin], ticlopidine, clopidogrel, abciximab, dextrans; corticosteroids, such as alclometasone, amcinonide, augmented betamethasone, beclomethasone, betamethasone, budesonide, cortisone, clobetasol, clocortolone, desonide, desoximetasone, dexamethasone, fluocinolone, fluocinonide, flurandrenolide, flunisolide, fluticasone, halcinonide, halobetasol, hydrocortisone, methylprednisolone, mometasone, prednicarbate, prednisone, prednisolone, triamcinolone; so-called non-steroidal anti-inflammatory drugs (NSAIDs), such as diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib; cytostatics, such as alkaloides and podophyllum toxins, such as vinblastine, vincristine; alkylating agents, such as nitrosoureas, nitrogen lost analogs; cytotoxic antibiotics, such as daunorubicin, doxorubicin and other anthracyclines and related substances, bleomycin, mitomycin; antimetabolites, such as folic acid analogs, purine analogs or pyrimidine analogs; paclitaxel, docetaxel, sirolimus; platinum compounds, such as carboplatin, cisplatin or oxaliplatin; amsacrin, irinotecan, imatinib, topotecan, interferon-alpha 2a, interferon-alpha 2b, hydroxycarbamide, miltefosine, pentostatin, porfimer, aldesleukin, bexaroten, tretinoin; antiandrogens and antiestrogens; antiarrythmics in particular class I antiarrhythmic, such as antiarrhythmics of the quinidine type, quinidine, dysopyramide, ajmaline, prajmalium bitartrate, detajmium bitartrate; antiarrhythmics of the lidocaine type, e.g., lidocaine, mexiletin, phenyloin, tocainid; class Ic antiarrhythmics, e.g., propafenon, flecainid(acetate); class II antiarrhythmics beta-receptor blockers, such as metoprolol, esmolol, propranolol, metoprolol, atenolol, oxprenolol; class III antiarrhythmics, such as amiodarone, sotalol; class IV antiarrhythmics, such as diltiazem, verapamil, gallopamil; other antiarrhythmics, such as adenosine, orciprenaline, ipratropium bromide; agents for stimulating angiogenesis in the myocardium, such as vascular endothelial growth factor (VEGF), basic fibroblast growth factor (bFGF), non-viral DNA, viral DNA, endothelial growth factors: FGF-1, FGF-2, VEGF, TGF; antibiotics, monoclonal antibodies, anticalins; stem cells, endothelial progenitor cells (EPC); digitalis glycosides, such as acetyl digoxin/metildigoxin, digitoxin, digoxin; cardiac glycosides, such as ouabain, proscillaridin; antihypertensives, such as CNS active antiadrenergic substances, e.g., methyldopa, imidazoline receptor agonists; calcium channel blockers of the dihydropyridine type, such as nifedipine, nitrendipine; ACE inhibitors: quinaprilate, cilazapril, moexipril, trandolapril, spirapril, imidapril, trandolapril; angiotensin II antagonists: candesartancilexetil, valsartan, telmisartan, olmesartanmedoxomil, eprosartan; peripherally active alpha-receptor blockers, such as prazosin, urapidil, doxazosin, bunazosin, terazosin, indoramin; vasodilatators, such as dihydralazine, diisopropylamine dichloracetate, minoxidil, nitroprusside sodium; other antihypertensives, such as indapamide, co-dergocrine mesylate, dihydroergotoxin methanessulfonate, cicletanin, bosentan, fludrocortisone; phosphodiesterase inhibitors, such as milrinon, enoximon and antihypotensives, such as in particular adrenergic and dopaminergic substances, such as dobutamine, epinephrine, etilefrine, norfenefrine, norepinephrine, oxilofrine, dopamine, midodrine, pholedrine, ameziniummetil; and partial adrenoceptor agonists, such as dihydroergotamine; fibronectin, polylysine, ethylene vinyl acetate, inflammatory cytokines, such as: TGF, PDGF, VEGF, bFGF, TNF, NGF, GM-CSF, IGF-a, IL-1, IL 8, IL-6, growth hormone; as well as adhesive substances, such as cyanoacrylates, beryllium, silica; and growth factors, such as erythropoetin, hormones, such as corticotropins, gonadotropins, somatropins, thyrotrophins, desmopressin, terlipressin, pxytocin, cetrorelix, corticorelin, leuprorelin, triptorelin, gonadorelin, ganirelix, buserelin, nafarelin, goserelin, as well as regulatory peptides, such as somatostatin, octreotid; bone and cartilage stimulating peptides, bone morphogenetic proteins (BMPs), in particulary recombinant BMPs, such as recombinant human BMP-2 (rhBMP-2), bisphosphonate (e.g., risedronate, pamidronate, ibandronate, zoledronic acid, clodronsaure, etidronsaure, alendronic acid, tiludronic acid), fluorides, such as disodium fluorophosphate, sodium fluoride; calcitonin, dihydrotachystyrol; growth factors and cytokines such as epidermal growth factor (EGF), platelet-derived growth factor (PDGF), fibroblast growth factors (FGFs), transforming growth factors-b (TGFs-b), transforming growth factor-a (TGF-a), erythropoietin (EPO), insulin-like growth factor-I (IGF-I), insulin-like growth factor-II (IGF-II), interleukin-1 (IL-1), interleukin-2 (IL-2), interleukin-6 (IL-6), interleukin-8 (IL-8), tumor necrosis factor-a (TNF-a), tumor necrosis factor-b (TNF-b), interferon-g (INF-g), colony stimulating factors (CSFs); monocyte chemotactic protein, fibroblast stimulating factor 1, histamine, fibrin or fibrinogen, endothelin-1, angiotensin II, collagens, bromocriptine, methysergide, methotrexate, carbon tetrachloride, thioacetamide and ethanol; as well as silver (ions), titanium dioxide, antibiotics and anti-infective drugs such as in particular β-lactam antibiotics, e.g., β-lactamase-sensitive penicillins such as benzyl penicillins (penicillin G), phenoxymethylpenicillin (penicillin V); β-lactamase-resistent penicillins, such as aminopenicillins, e.g., amoxicillin, ampicillin, bacampicillin; acylaminopenicillins, such as mezlocillin, piperacillin; carboxypenicillins, cephalosporins such as cefazoline, cefuroxim, cefoxitin, cefotiam, cefaclor, cefadroxil, cefalexin, loracarbef, cefixim, cefuroximaxetil, ceftibuten, cefpodoximproxetil, cefpodoximproxetil; aztreonam, ertapenem, meropenem; βm-lactamase inhibitors, such as sulbactam, sultamicillintosylate; tetracyclines, such as doxycycline, minocycline, tetracycline, chlorotetracycline, oxytetracycline; aminoglycosides, such as gentamicin, neomycin, streptomycin, tobramycin, amikacin, netilmicin, paromomycin, framycetin, spectinomycin; macrolide antibiotics, such as azithromycin, clarithromycin, erythromycin, roxithromycin, spiramycin, josamycin; lincosamides, such as clindamycin, lincomycin; gyrase inhibitors such as fluoroquinolones, e.g., ciprofloxacin, ofloxacin, moxifloxacin, norfloxacin, gatifloxacin, enoxacin, fleroxacin, levofloxacin; quinolones, such as pipemidic acid; sulfonamides, trimethoprim, sulfadiazine, sulfalene; glycopeptide antibiotics such as vancomycin, teicoplanin; polypeptide antibiotics, such as polymyxins, e.g., colistin, polymyxin-b, nitroimidazole derivates, e.g., metronidazole, timidazole; aminoquinolones, such as chloroquin, mefloquin, hydroxychloroquin; biguanids such as proguanil; quinine alkaloids and diaminopyrimidines, such as pyrimethamine; amphenicols, such as chloramphenicol; rifabutin, dapson, fusidic acid, fosfomycin, nifuratel, telithromycin, fusafungin, fosfomycin, pentamidine diisethionate, rifampicin, taurolidin, atovaquon, linezolid; virus static, such as aciclovir, ganciclovir, famciclovir, foscamet, inosine-(dimepranol-4-acetamidobenzoate), valganciclovir, valaciclovir, cidofovir, brivudin; antiretroviral active ingredients (nucleoside analog reverse-transcriptase inhibitors and derivatives), such as lamivudine, zalcitabine, didanosine, zidovudin, tenofovir, stavudin, abacavir; non-nucleoside analog reverse-transcriptase inhibitors: amprenavir, indinavir, saquinavir, lopinavir, ritonavir, nelfinavir; amantadine, ribavirine, zanamivir, oseltamivir or lamivudine, as well as any combinations and mixtures thereof.

In another exemplary embodiment of the present invention, the active agents are encapsulated in polymers, vesicles, liposomes or micelles.

Suitable exemplary diagnostically active agents for use in the present invention can be e.g., signal generating agents or materials, which may be used as markers. Such signal generating agents include materials which in physical, chemical and/or biological measurement and verification methods lead to detectable signals, for example in image-producing methods. It is not important for the present invention, whether the signal processing is carried out exclusively for diagnostic or therapeutic purposes. Typical imaging methods are for example radiographic methods, which are based on ionizing radiation, for example conventional X-ray methods and X-ray based split image methods, such as computer tomography, neutron transmission tomography, radiofrequency magnetization, such as magnetic resonance tomography, further by radionuclide-based methods, such as scintigraphy, Single Photon Emission Computed Tomography (SPECT), Positron Emission Computed Tomography (PET), ultrasound-based methods or fluoroscopic methods or luminescence or fluorescence based methods, such as Intravasal Fluorescence Spectroscopy, Raman spectroscopy, Fluorescence Emission Spectroscopy, Electrical Impedance Spectroscopy, colorimetry, optical coherence tomography, etc, further Electron Spin Resonance (ESR), Radio Frequency (RF) and Microwave Laser and similar methods.

Exemplary signal generating agents can be metal-based from the group of metals, metal oxides, metal carbides, metal nitrides, metal oxynitrides, metal carbonitrides, metal oxycarbides, metal oxynitrides, metal oxycarbonitrides, metal hydrides, metal alkoxides, metal halides, inorganic or organic metal salts, metal polymers, metallocenes, and other organometallic compounds, chosen from powders, solutions, dispersions, suspensions, emulsions. Preferred metal based agents are especially nanomorphous nanoparticles from metals, metal oxides or mixtures there from. The metals or metal oxides used can also be magnetic; examples are—without excluding other metals—iron, cobalt, nickel, manganese or mixtures thereof, for example iron-platinum mixtures, or as an example for magnetic metal oxides, iron oxide and ferrites.

It can be preferred to use semi conducting nanoparticles, examples for this are semiconductors from group II-VI, group III-V, group IV. Group II-VI—semiconductors are for example MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe or mixtures thereof. Examples of group III-V semiconductors are for example GaAs, GaN, GaP, GaSb, InGaAs, InP, InN, InSb, InAs, AlAs, AlP, AlSb, AlS, and mixtures thereof are preferred. Germanium, lead and silicon are selected as exemplary of group IV semiconductors. The semiconductors can moreover also contain mixtures of semiconductors from more than one group, all groups mentioned above are included.

It can moreover be preferred to choose complex formed metal-based nanoparticles. Included here are so-called Core-Shell configurations, as described explicitly by Peng et al., “Epitaxial Growth of Highly Luminescent CdSe/CdS Core/Shell Nanoparticles with Photo stability and Electronic Accessibility”, Journal of the American Chemical Society, (1997) 119:7019-7029, and included herewith explicitly per reference. Preferred here are semi conducting nanoparticles, which form a core with a diameter of 1-30 nm, especially preferred of about 1-15 nm, onto which other semi conducting nanoparticles crystallize in 1-50 monolayers, further preferred can be about 1-15 monolayers. In this case core and shell can be present in any desired combinations as described above, in special embodiments CdSe and CdTe ma be preferred as the core and CdS and ZnS as the shell.

Further, exemplary signal producing metal-based agents can be selected from salts or metal ions, which preferably have paramagnetic properties, for example lead (II), bismuth (II), bismuth (III), chromium (III), manganese (II), manganese (III), iron (II), iron (III), cobalt (II), nickel (II), copper (II), praseodymium (III), neodymium (III), samarium (III), or ytterbium (III), holmium (III) or erbium (III) and the like. Based on especially pronounced magnetic moments, especially gadolinium (III), terbium (III), dysprosium (III), holmium (III) and erbium (III) are mostly preferred. Further one can select from radioisotopes. Examples of a few applicable radioisotopes include H 3, Be 1, O 15, Ca 49, Fe 60, In 111, Pb 210, Ra 220, Ra 224 and the like. Typically such ions are present as chelates or complexes, wherein for example as chelating agents or ligands for lanthanides and paramagnetic ions compounds, such as diethylenetriamine pentaacetic acid (“DTPA”), ethylenediamine tetra acetic acid (“EDTA”), or tetraazacyclododecane-N,N′,N″,N′″-tetra acetic acid (“DOTA”) are used. Other typical organic complexing agents are for example published in Alexander, Chem. Rev. 95:273-342 (1995) and Jackels, Pharm. Med. Imag, Section III, Chap. 20, p645 (1990). Other usable chelating agents in the present invention, are found in U.S. Pat. Nos. 5,155,215, 5,087,440, 5,219,553, 5,188,816, 4,885,363, 5,358,704, 5,262,532, 5,188,816, 4,885,363, and 5,219,553 and Meyer et al., Invest. Radiol. 25: S53 (1990). Preferred mostly are salts and chelates from the lanthanide group with the atomic numbers 57-83 or the transition metals with the atomic numbers 21-29, or 42 or 44.

Especially preferred are paramagnetic perfluoroalkyl containing compounds which for example are described in German laid-open patents German Patent Publications DE 196 03 033, DE 197 29 013 and in International Publication WO 97/26017, further diamagnetic perfluoroalkyl containing substances of the general formula R<PF>-L<II>-G<III>, wherein R<PF> represents a perfluoroalkyl group with 4 to 30 carbon atoms, L<II> stands for a linker and G<III> for a hydrophilic group. The linker L is a direct bond, an —SO₂-group or a straight or branched carbon chain with up to 20 carbon atoms which can be substituted with one or more —OH, —COO<−>, —SO₃-groups and/or if necessary one or more —O—, —S—, —CO—, —CONH—, —NHCO—, —CONR—, —NRCO—, —SO2-, —PO4-, —NH—, —NR-groups, an aryl ring or contain a piperazine, wherein R stands for a C₁ to C₂₀ alkyl group, which again can contain and/or have one or a plurality of O atoms and/or be substituted with —COO<−> or SO₃-groups.

The hydrophilic group G<III> can be selected from a mono or disaccharide, one or a plurality of —COO<−> or —SO₃<−>-groups, a dicarboxylic acid, an isophthalic acid, a picolinic acid, a benzenesulfonic acid, a tetrahydropyranedicarboxylic acid, a 2,6-pyridinedicarboxylic acid, a quaternary ammonium ion, an aminopolycarboxcylic acid, an aminodipolyethyleneglycol sulfonic acid, an aminopolyethyleneglycol group, an SO₂—(CH₂)₂—OH-group, a polyhydroxyalkyl chain with at least two hydroxyl groups or one or a plurality of polyethylene glycol chains having at least two glycol units, wherein the polyethylene glycol chains are terminated by an —OH or —OCH₃— group, or similar linkages. See, for example, published German Patent Application DE 199 48 651, incorporated herein by reference in its entirety.

It can be preferred in further exemplary embodiments to select paramagnetic metals in the form of metal complexes with phthalocyanines, especially as described in Phthalocyanine Properties and Applications, Vol. 14, C. C. Leznoff and A. B. P. Lever, V C H Ed., wherein as examples to mention are octa(1,4,7,10-tetraoxaundecyl)Gd-phthalocyanine, octa(1,4,7,10-tetraoxaundecyl)Gd-phthalocyanine, octa(1,4,7,10-tetraoxaundecyl)Mn-phthalocyanine, octa(1,4,7,10-tetraoxaundecyl)Mn-phthalocyanine, as described in U.S. Patent Publication No. 2004/214810.

It can further be preferred to select from super-paramagnetic, ferromagnetic or ferrimagnetic signal generating agents. For example among magnetic metals, alloys are preferred, among ferrites like gamma iron oxide, magnetites or cobalt-, nickel- or manganese-ferrites, corresponding agents are preferably selected, especially particles as described in International Publication Nos. WO83/03920, WO83/01738, WO85/02772, WO89/03675, WO88/00060 and WO90/01899, in U.S. Pat. Nos. 4,452,773, 4,675,173 and 4,770,183.

Further, magnetic, paramagnetic, diamagnetic or super paramagnetic metal oxide crystals having diameters of less than about 4000 Angstroms are especially preferred as degradable non-organic agents. Suitable metal oxides can be selected from iron oxide, cobalt oxides, iridium oxides or the like, which provide suitable signal producing properties and which have especially biocompatible properties or are biodegradable. It can also preferable that crystalline agents of this group can have diameters smaller than about 500 Angstroms. These crystals can be associated covalently or non-covalently with macromolecular species and are modified, such as the metal-based signal generating agents described above.

Further, zeolite containing paramagnets and gadolinium containing nanoparticles are selected from polyoxometallates, preferably of the lanthanides, (e.g., K9GdW10036).

It is preferred to limit the average particle size of the magnetic signal producing agents to maximal 5 μm in order to optimize the image producing properties, and it is especially preferred that the magnetic signal producing particles be of a size from 2 nm up to 1 μm, most preferably 5 nm to 200 nm. The super paramagnetic signal producing agents can be chosen for example from the group of so-called SPIOs (super paramagnetic iron oxides) with a particle size larger than 50 nm or from the group of the USPIOs (ultra small super paramagnetic iron oxides) with particle sizes smaller than 50 nm.

In accordance with the present invention it can be preferred to select signal generating agents from the group of endohedral fullerenes, as disclosed for example in U.S. Pat. No. 5,688,486 or International Publication No. WO 93/15768, which are incorporated by reference. It is further preferred to select fullerene derivatives and their metal complexes. Especially preferred are fullerene species, which comprise carbon clusters having 60, 70, 76, 78, 82, 84, 90, 96 or more carbon atoms. An overview of such species can be gathered from European Patent Publication 1331226 and is incorporated herein by reference in its entirety.

Further metal fullerenes or endohedral carbon-carbon nanoparticles with arbitrary metal-based components can also be selected. Such endohedral fullerenes or endometallo fullerenes can be preferred, which for example contain rare earths, such as cerium, neodymium, samarium, europium, gadolinium, terbium, dysprosium or holmium. Moreover it can be especially preferred to use carbon coated metallic nanoparticles, such as carbides. The choice of nanomorphous carbon species is not limited to fullerenes, since it can be preferred to select from other nanomorphous carbon species, such as nanotubes, onions, etc. In another exemplary embodiment, it can be preferred to select fullerene species from non-endohedral or endohedral forms, which contain halogenated, preferably iodated, groups, as described in U.S. Pat. No. 6,660,248.

In certain exemplary embodiments mixtures of such signal generating agents of different specifications are also used, depending on the desired properties of the wanted signal generating material properties. The signal producing agents used generally can have a size of about 0.5 nm to 1000 nm, preferably about 0.5 nm to 900 nm, especially preferably from about 0.7 to 100 nm. In this connection the metal-based nanoparticles can be provided as a powder, in polar, non-polar or amphiphilic solutions, dispersions, suspensions or emulsions. Nanoparticles are easily modifiable based on their large surface to volume ratios. The nanoparticles to be selected can for example be modified non-covalently by means of hydrophobic ligands, for example with trioctylphosphine, or be covalently modified. Examples of covalent ligands are thiol fatty acids, amino fatty acids, fatty acid alcohols, fatty acids, fatty acid ester groups or mixtures thereof, for example oleic cid and oleylamine.

In accordance with certain exemplary embodiments of the present invention, the signal producing agents can be encapsulated in micelles or liposomes with the use of amphiphilic components, or may be encapsulated in polymeric shells, wherein the micelles/liposomes can have a diameter of about 2 nm to 800 nm, preferably from about 5 to 200 nm, further preferably from about 10 to 25 nm. The size of the micelles/liposomes is, without committing to a specific theory, dependant on the number of hydrophobic and hydrophilic groups, the molecular weight of the nanoparticles and the aggregation number. In aqueous solutions the use of branched or unbranched amphiphilic substances, is especially preferred in order to achieve the encapsulation of signal generating agents in liposomes/micelles. The hydrophobic nucleus of the micelles hereby contains in a further exemplary embodiment, a multiplicity of hydrophobic groups, preferably between 1 and 200, especially preferred between about 1 and 100 and further preferably between about 1 and 30 according to the desired setting of the micelle size.

Exemplary hydrophobic groups consist preferably of hydrocarbon groups or residues or silicon-containing residues, for example polysiloxane chains. Furthermore, they can preferably be selected from hydrocarbon-based monomers, oligomers and polymers, or from lipids or phospholipids or comprise combinations hereof, especially glyceryl esters, such as phosphatidyl ethanolamine, phosphatidyl choline, or polyglycolides, polylactides, polymethacrylate, polyvinylbutylether, polystyrene, polycyclopentadienylmethylnorbornene, polyethylenepropylene, polyethylene, polyisobutylene, polysiloxane. Further for encapsulation in micelles hydrophilic polymers are also selected, especially preferred polystyrenesulfonic acid, poly-N-alkylvinylpyridiniumhalides, poly(meth)acrylic acid, polyamino acids, poly-N-vinylpyrrolidone, polyhydroxyethylmethacrylate, polyvinyl ether, polyethylene glycol, polypropylene oxide, polysaccharides, such as agarose, dextrane, starches, cellulose, amylose, amylopectin, or polyethylene glycol or polyethylene imine of any desired molecular weight, depending on the desired micelles property. Further, mixtures of hydrophobic or hydrophilic polymers can be used or such lipid-polymer compositions employed. In a further special embodiment, the polymers are used as conjugated block polymers, wherein hydrophobic and also hydrophilic polymers or any desired mixtures there of can be selected as 2-, 3- or multi-block copolymers.

Such exemplary signal generating agents encapsulated in micelles can moreover be functionalized, while linker (groups) are attached at any desired position, preferably amino-, thiol, carboxyl-, hydroxyl-, succinimidyl, maleimidyl, biotin, aldehyde- or nitrilotriacetate groups, to which any desired corresponding chemically covalent or non-covalent other molecules or compositions can be bound according to the conventional. Here, especially biological molecules, such as proteins, peptides, amino acids, polypeptides, lipoproteins, glycosaminoglycanes, DNA, RNA or similar bio molecules are preferred especially.

It can be preferable to select signal generating agents from non-metal-based signal generating agents, for example from the group of X-ray contrast agents, which can be ionic or non-ionic. Among the ionic contrast agents are included salts of 3-acetyl amino-2,4-6-triiodobenzoic acid, 3,5-diacetamido-2,4,6-triiodobenzoic acid, 2,4,6-triiodo-3,5-dipropionamido-benzoic acid, 3-acetyl amino-5-((acetyl amino)methyl)-2,4,6-triiodobenzoic acid, 3-acetyl amino-5-(acetyl methyl amino)-2,4,6-triiodobenzoic acid, 5-acetamido-2,4,6-triiodo-N-((methylcarbamoyl)methyl)-isophthalamic acid, 5-(2-methoxyacetamido)-2,4,6-triiodo-N-[2-hydroxy-1-(methylcarbamoyl)-ethoxy l]-isophthalamic acid, 5-acetamido-2,4,6-triiodo-N-methylisophthalamic acid, 5-acetamido-2,4,6-triiodo-N-(2-hydroxyethyl)-isophthalamic acid 2-[[2,4,6-triiodo-3-[(1-oxobutyl)-amino]phenyl]methyl]-butanoic acid, beta-(3-amino-2,4,6-triiodophenyl)-alpha-ethyl-propanoic acid, 3-ethyl-3-hydroxy-2,4,6-triiodophenyl-propanoic acid, 3-[[(dimethylamino)-methyl]amino]-2,4,6-triiodophenyl-propanoic acid (see Chem. Ber. 93: 2347 (1960)), alpha-ethyl-(2,4,6-triiodo-3-(2-oxo-1-pyrrolidinyl)-phenyl)-propanoic acid, 2-[2-[3-(acetyl amino)-2,4,6-triiodophenoxy]ethoxymethyl]butanoic acid, N-(3-amino-2,4,6-triiodobenzoyl)-N-phenyl-.beta.-aminopropanoic acid, 3-acetyl-[(3-amino-2,4,6-triiodophenyl)amino]-2-methylpropanoic acid, 5-[(3-amino-2,4,6-triiodophenyl)methyl amino]-5-oxypentanoic acid, 4-[ethyl-[2,4,6-triiodo-3-(methyl amino)-phenyl]amino]-4-oxo-butanoic acid, 3,3′-oxy-bis[2,1-ethanediyloxy-(1-oxo-2,1-ethanediyl)imino]bis-2,4,6-triiodobenzoic acid, 4,7,10,13-tetraoxahexadecane-1,16-dioyl-bis(3-carboxy-2,4,6-triiodoanilide), 5,5′-(azelaoyldiimino)-bis[2,4,6-triiodo-3-(acetyl amino)methyl-benzoic acid], 5,5′-(apidoldiimino)bis(2,4,6-triiodo-N-methyl-isophthalamic acid), 5,5′-(sebacoyl-diimino)-bis(2,4,6-triiodo-N-methylisophthalamic acid), 5,5-[N,N-diacetyl-(4,9-dioxy-2,11-dihydroxy-1,12-dodecanediyl)diimino]bis(2,4,6-triiodo-N-methyl-isophthalamic acid), 5,5′5″-(nitrilo-triacetyltriimino)tris(2,4,6-triiodo-N-methyl-isophthalamic acid), 4-hydroxy-3,5-diiodo-alpha-phenylbenzenepropanoic acid, 3,5-diiodo-4-oxo-1(4H)-pyridine acetic acid, 1,4-dihydro-3,5-diiodo-1-methyl-4-oxo-2,6-pyridinedicarboxylic acid, 5-iodo-2-oxo-1(2H)-pyridine acetic acid, and N-(2-hydroxyethyl)-2,4,6-triiodo-5-[2,4,6-triiodo-3-(N-methylacetamido)-5-(methylcarbomoyl)benzamino]acetamido]-isophthalamic acid, and the like, especially preferred, as well as other ionic X-ray contrast agents suggested in the literature, for example in J. Am. Pharm. Assoc., Sci. Ed. 42:721 (1953), Swiss Patent 480071, JACS 78:3210 (1956), German patent 2229360, U.S. Pat. Nos. 3,476,802, 2,705,726, 2,895,988, 2,551,696, 1,993,039 and 4,005,188, Arch. Pharm. (Weinheim, Germany) 306: 11 834 (1973), J. Med. Chem. 6: 24 (1963), FR-M-6777, Pharmazie 16: 389 (1961), Chem. Ber. 93:2347 (1960), SA-A-68/01614, Acta Radiol. 12: 882 (1972), British Patent 870321, Rec. Trav. Chim. 87: 308 (1968), East German Patent Publication Nos. 67209, 2050217 and 2405652, Farm Ed. Sci. 28: 912 (1973), Farm Ed. Sci. 28: 996 (1973), J. Med. Chem. 9: 964 (1966), Arzheim.-Forsch 14: 451 (1964), SE-A-344166, British Patent Publication 1346796, Ann 494: 284 (1932), and J. Pharm. Soc. (Japan) 50: 727 (1930).

Examples of applicable non-ionic X-ray contrast agents in accordance with the exemplary embodiments of the present invention, are metrizamide as described in DE-A-2031724, iopamidol as described in BE-A-836355, iohexyl as disclosed in GB-A-1548594, iotrolan as described in EP-A-33426, iodecimol as described in EP-A-49745, iodixanol as in EP-A-108638, ioglucol as described in U.S. Pat. No. 4,314,055, ioglucomide as described in BE-A-846657, ioglunioe as described in DE-A-2456685, iogulamide as described in BE-A-882309, iomeprol as described in EP-A-26281, iopentol as described in EP-A-105752, iopromide as described in DE-A-2909439, iosarcol as described in DE-A-3407473, iosimide as described in DE-A-3001292, iotasul as described in EP-A-22056, iovarsul as disclosed in EP-A-83964 or ioxilan described in WO87/00757, and the like.

In certain exemplary embodiments, it is possible to select agents based on nanoparticle signal generating agents, which after release into tissues and cells are incorporated or are enriched in intermediate cell compartments and/or have an especially long residence time in the organism. Such particles are selected in a special embodiment from water-insoluble agents, in another exemplary embodiment, they contain a heavy element, such as iodine or barium, in a third PH-50 as monomer, oligomer or polymer (iodinated aroyloxy ester having the empirical formula C₁₉H₂₃I₃N₂O₆, and the chemical names 6-ethoxy-6-oxohexy-3,5-bis(acetyl amino)-2,4,6-triiodobenzoate), in a fourth embodiment, an ester of diatrizoic acid, in a fifth an iodinated aroyloxy ester or in a sixth embodiment, any combinations hereof. In these embodiments particle sizes are preferred, which can be incorporated by macrophages. A corresponding method for this is disclosed in WO03039601 and agents preferred to be selected are disclosed in the publications U.S. Pat. Nos. 5,322,679, 5,466,440, 5,518,187, 5,580,579, and 5,718,388, gel of which are explicitly incorporated by reference in accordance with the present invention. Further advantageous are particularly, nanoparticles which are marked with signal generating agents or such signal generating agents, such as PH-50, which accumulate in intercellular spaces and can make interstitial as well as extrastitial compartments visible.

Signal generating agents can be selected moreover from the group of the anionic or cationic lipids, as disclosed already in U.S. Pat. No. 6,808,720 and explicitly incorporated herewith. Further preferred are anionic lipids, such as phosphatidyl acid, phosphatidyl glycerol and their fatty acid esters, or amides of phosphatidyl ethanolamine, such as anandamide and methanandamide, phosphatidyl serine, phosphatidyl inositol and their fatty acid esters, cardiolipin, phosphatidyl ethylene glycol, acid lysolipids, palmitic acid, stearic acid, arachidonic acid, oleic acid, linoleic acid, linolenic acid, myristic acid, sulfolipids and sulfatides, free fatty acids, both saturated and unsaturated and their negatively charged derivatives, and the like. Moreover, specially halogenated, in particular fluorinated anionic lipids are preferred. The anionic lipids can contain cations from the alkaline earth metals beryllium (Be<+2>), magnesium (Mg<+2>), calcium (Ca<+2>), strontium (Sr<+2>) and barium (Ba<+2>), or amphoteric ions, such as aluminium (Al<+3>), gallium (Ga<+3>), germanium (Ge<+3>), tin (Sn+<4>) or lead (Pb<+2> and Pb<+4>), or transition metals, such as titanium (Ti<+3> and Ti<+4>), vanadium (V<+2> and V<+3>), chromium (Cr<+2> and Cr<+3>), manganese (Mn<+2> and Mn<+3>), iron (Fe<+2> and Fe<+3>), cobalt (Co<+2> and Co<+3>), nickel (Ni<+2> and Ni<+3>), copper (Cu<+2>), zinc (Zn<+2>), zirconium (Zr<+4>), niobium (Nb<+3>), molybdenum (Mo<+2> and Mo<+3>), cadmium (Cd<+2>), indium (In <+3>), tungsten (W<+2> and W<+4>), osmium (Os<+2>, Os<+3> and Os<+4>), iridium (Ir<+2>, Ir<+3> and Ir<+4>), mercury (Hg<+2>) or bismuth (Bi<+3>), and/or rare earths, such as lanthanides, for example lanthanum (La<+3>) and gadolinium (Gd<+3>). Especially preferred cations are calcium (Ca<+2>), magnesium (Mg<+2>) and zinc (Zn<+2>) and paramagnetic cations, such as manganese (Mn<+2>) or gadolinium (Gd<+3>).

Cationic lipids can be chosen from phosphatidyl ethanolamine, phospatidylcholine, Glycero-3-ethylphosphatidylcholine and their fatty acid esters, di- and tri-methylammoniumpropane, di- and tri-ethylammoniumpropane and their fatty acid esters. Especially preferred derivatives are N-[1-(2,3-dioleoyloxy)propyl]-N,N,N-trimethylammonium chloride (“DOTMA”). Furthermore synthetic cationic lipids based on for example naturally occurring lipids, such as dimethyldioctadecyl-ammonium bromide, sphingolipids, sphingomyelin, lysolipids, glycolipids such as for example gangliosides GM1, sulfatides, glycosphingolipids, cholesterol and cholesterol esters or salts, N-succinyldioleoylphosphattidyl ethanolamine, 1,2-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidyl ethanolamine and palmitoyl-homocystein, mostly preferred are fluorinated, derivatized cationic lipids. Such compounds have been described especially in U.S. application Ser. No. 08/391,938.

Such exemplary lipids are furthermore suitable as components of signal generating liposomes, which especially can have pH— sensitive properties as disclosed in U.S. Publication No. 2004/197392.

In accordance with certain exemplary embodiments of the present invention, signal generating agents can also be selected from the group of the so-called microbubbles or microballoons, which contain stable dispersions or suspensions in a liquid carrier substance. Gases to be chosen are preferably air, nitrogen, carbon dioxide, hydrogen or noble gases, such as helium, argon, xenon or krypton, or sulfur-containing fluorinated gases, such as sulfurhexafluoride, disulfurdecafluoride or trifluoromethylsulfurpentafluoride, or for example selenium hexafluoride, or halogenated silanes, such as methylsilane or dimethylsilane, further short chain hydrocarbons, such as alkanes, specifically methane, ethane, propane, butane or pentane, or cycloalkanes, such as cyclopropane, cyclobutane or cyclopentane, also alkenes, such as ethylene, propene, propadiene or butene, or also alkynes, such as acetylene or propyne. Further ethers, such as dimethylether can be considered or be chosen, or ketones, or esters or halogenated short-chain hydrocarbons, or any desired mixtures of the above. Especially preferred are halogenated or fluorinated hydrocarbon gases, such as bromochlorodifluoromethane, chlorodifluoromethane, dichlorodifluoromethane, bromotrifluoromethane, chlorotrifluoromethane, chloropentafluoroethane, dichlorotetrafluoroethane, chlorotrifluoroethylene, fluoroethylene, ethyl fluoride, 1,1-difluoroethane or perfluorohydrocarbons, such as, for example, perfluoroalkanes, perfluorocycloalkanes, perfluoroalkenes or perfluorinated alkynes. Especially preferred are emulsions of liquid dodecafluoropentane or decafluorobutane and sorbitol, or similar, as described in International Publication No. WO93/05819 and explicitly incorporated herewith by reference.

Preferably such microbubbles are selected, which are encapsulated in compounds having the structure R1-X-Z; R2-X-Z; or R3-X-Z′, wherein R1, R2 comprises and R3 hydrophobic groups selected from straight chain alkylenes, alkyl ethers, alkyl thiol ethers, alkyl disulfides, polyfluoroalkylenes and polyfluoroalkylethers, Z comprises a polar group from CO₂-M<+>, SO₃<−> M<+>, SO4<−> M<+>, PO₃<−> M<+>, PO₄<−> M<+2>, N(R)₄<+> or a pyridine or substituted pyridine, and a zwitterionic group, M is a metal ion, and finally X represents a linker which binds the polar group with the residues.

Gas-filled or in situ out-gassing micro spheres having a size of less than 1000 μm can be further selected from biocompatible synthetic polymers or copolymers which comprise monomers, dimers or oligomers or other pre-polymer to pre-stages of the following polymerizable substances: acrylic acid, methacrylic acid, ethyleneimine, crotonic acid, acryl amide, ethyl acrylate, methylmethacrylate, 2-hydroxyethylmethacrylate (HEMA), lactonic acid, glycolic acid, [epsilon]caprolactone, acrolein, cyanoacrylate, bisphenol A, epichlorhydrin, hydroxyalkylacrylate, siloxane, dimethylsiloxane, ethylene oxide, ethylene glycol, hydroxyalkylmethacrylate, N-substituted acryl amide, N-substituted methacrylamides, N-vinyl-2-pyrrolidone, 2,4-pentadiene-1-ol, vinyl acetate, acrylonitrile, styrene, p-aminostyrene, p-aminobenzylstyrene, sodium styrenesulfonate, sodium-2-sulfoxyethylmethacrylate, vinyl pyridine, aminoethylmethacrylate, 2-methacryloyloxytrimethylammonium chloride, and polyvinylidenes, such as polyfunctional cross-linkable monomers, such as for example N,N′-methylene-bis-acrylamide, ethylene glycol dimethacrylate, 2,2′-(p-phenylenedioxy)-diethyldimethacrylate, divinylbenzene, triallylamine and methylene-bis-(4-phenyl-isocyanate), including any desired combinations thereof. Preferred polymers contain polyacrylic acid, polyethyleneimine, polymethacrylic acid, polymethylmethacrylate, polysiloxane, polydimethylsiloxane, polylactonic acid, poly([epsilon]-caprolactone), epoxy resins, poly(ethylene oxide), poly(ethylene glycol), and polyamides (e.g., Nylon) and the like, or any arbitrary mixtures thereof. Preferred copolymers contain among others polyvinylidene-polyacrylonitrile, polyvinylidene-polyacrylonitrile-polymethylmethacrylate, and polystyrene-polyacrylonitrile and the like, or any desired mixtures thereof. Methods for manufacture of such micro spheres are published for example in U.S. Pat. No. 4,179,546, U.S. Pat. No. 3,945,956, U.S. Pat. No. 4,108,806, Japan Kokai Tokkyo Koho 62 286534, British Patent No. 1,044,680, U.S. Pat. No. 3,293,114, U.S. Pat. No. 3,401,475, U.S. Pat. No. 3,479,811, U.S. Pat. No. 3,488,714, U.S. Pat. No. 3,615,972, U.S. Pat. No. 4,549,892, U.S. Pat. No. 4,540,629, U.S. Pat. No. 4,421,562, U.S. Pat. No. 4,420,442, U.S. Pat. No. 4,898,734, U.S. Pat. No. 4,822,534, U.S. Pat. No. 3,732,172, U.S. Pat. No. 3,594,326, U.S. Pat. No. 3,015,128, Deasy, Microencapsulation and Related Drug Processes, Vol. 20, Chapters. 9 and 10, pp. 195-240 (Marcel Dekker, Inc., N.Y., 1984), Chang et al., Canadian J of Physiology and Pharmacology, Vol 44, pp. 115-129 (1966), and Chang, Science, Vol. 146, pp. 524-525 (1964).

Other exemplary signal generating agents can in accordance with the present invention be selected from the group of agents, which are transformed into signal generating agents in organisms by means of in-vitro or in-vivo cells, cells as a component of cell cultures, of in-vitro tissues, or cells as a component of multicellular organisms, such as for example fungi, plants or animals, in further exemplary embodiments from mammals, such as mice or humans. Such exemplary agents can be made available in the form of vectors for the transfection of multicellular organisms, wherein the vectors contain recombinant nucleic acids for the coding of signal generating agents. In certain exemplary embodiments this is concerned with signal generating agents, such as metal binding proteins. It can be preferred to choose such vectors from the group of viruses for example from adeno viruses, adeno virus associated viruses, herpes simplex viruses, retroviruses, alpha viruses, pox viruses, arena-viruses, vaccinia viruses, influenza viruses, polio viruses or hybrids of any of the above.

Further such signal generating agents are to be chosen in combination with delivery systems, in order to incorporate nucleic acids, which are suitable for coding for signal generating agents, into the target structure. Further preferred are virus particles for the transfection of mammalian cells, wherein the virus particle contains one or a plurality of coding sequence/s for one or a plurality of signal generating agents as described above. In these cases the particles are generated from one or a plurality of the following viruses: adeno viruses, adeno virus associated viruses, herpes simplex viruses, retroviruses, alpha viruses, pox viruses, arena-viruses, vaccinia viruses, influenza viruses and polio viruses.

In further exemplary embodiments, these signal generating agents are made available from colloidal suspensions or emulsions, which are suitable to transfect cells, preferably mammalian cells, wherein these colloidal suspensions and emulsions contain those nucleic acids which possess one or a plurality of the coding sequence(s) for signal generating agents. Such colloidal suspensions or emulsions can contain macromolecular complexes, nano capsules, microspheres, beads, micelles, oil-in-water- or water-in-oil emulsions, mixed micelles and liposomes or any desired mixture of the above.

In further exemplary embodiments, cells, cell cultures, organized cell cultures, tissues, organs of desired species and non-human organisms can be chosen which contain recombinant nucleic acids having coding sequences for signal generating agents. In certain exemplary embodiments organisms are selected from the groups: mouse, rat, dog, monkey, pig, fruit fly, nematode worms, fish or plants or fungi. Further, cells, cell cultures, organized cell cultures, tissues, organs of desired species and non-human organisms can contain one or a plurality of vectors as described above.

Signal generating agents can be produced in vivo from the group of proteins and made available as described above. Such agents are preferably directly or indirectly signal producing, while the cells produce (direct) a signal producing protein through transfection or produce a protein which induces (indirect) the production of a signal producing protein. Preferably these signal generating agents are detectable in methods, such as MRI while the relaxation times T1, T2 or both are altered and lead to signal producing effects which can be processed sufficiently for imaging. Such proteins are preferably protein complexes, especially metalloprotein complexes. Direct signal producing proteins are preferably such metalloprotein complexes which are formed in the cells. Indirect signal producing agents are such proteins or nucleic acids, for example, which regulate the homeostasis of iron metabolism, the expression of endogenous genes for the production of signal generating agents, and/or the activity of endogenous proteins with direct signal generating properties, for example Iron Regulatory Protein (IRP), Transferrin receptor (for the take-up of Fe), erythroid-5-aminobevulinate synthase (for the utilization of Fe, H-Ferritin and L-Ferritin for the purpose of Fe storage). In certain exemplary embodiments, it can be preferred to combine both types of signal generating agents, that is direct and indirect, with each other, for example an indirect signal generating agent, which regulates the iron-homeostasis and a direct agent, which represents a metal binding protein.

In such embodiments, where preferably metal-binding polypeptides are selected as indirect agents, it is advantageous if the polypeptide binds to one or a plurality of metals which possess signal generating properties. Especially preferred are such metals with unpaired electrons in the Dorf orbitals, such as for example Fe, Co, Mn, Ni, Gd etc., wherein especially Fe is available in high physiological concentrations in organisms. It is moreover preferred, if such agents form metal-rich aggregates, for example crystalline aggregates, whose diameters are larger than 10 picometers, preferably larger than 100 picometers, 1 nm, 10 nm or specially preferred larger than 100 nm.

It may be preferred to include such metal-binding compounds, which have sub-nanomolar affinities with dissociation constants of less than 10⁻¹⁵ M, 10⁻² M or smaller. Typical polypeptides or metal-binding proteins are lactoferrin, ferritin, or other dimetallocarboxylate proteins or the like, or so-called metal catcher with siderophoric groups, such as for example haemoglobin. A possible exemplary method for preparation of such signal generating agents, their selection and the possible direct or indirect agents which are producible in vivo and are suitable as signal generating agents was disclosed in WO 03/075747 and is incorporated herewith in accordance with the present invention.

Another group of exemplary signal generating agents can be photophysically signal producing agents which consist of dyestuff-peptide-conjugates. Such dyestuff-peptide-conjugates are preferred which provide a wide spectrum of absorption maxima, for example polymethin dyestuffs, in particular cyanine-, merocyanine-, oxonol- and squarilium dyestuffs. From the class of the polymethin dyestuffs the cyanine dyestuffs, e.g., the indole structure based indocarbo-, indodicarbo- and indotricarbocyanines, are especially preferred. Such dyestuffs can be preferred in certain exemplary embodiments, which are substituted with suitable linking agents and can be functionalized with other groups as desired. In this connection see also German Patent Application DE 19917713.

In accordance with certain exemplary embodiments of the present invention, signal generating agents can be functionalized as desired. The functionalization by means of so-called “Targeting” groups is preferred are to be understood, as functional chemical compounds which link the signal generating agent or its specifically available form (encapsulation, micelles, micro spheres, vectors etc.) to a specific functional location, or to a determined cell type, tissue type or other desired target structures. Preferably targeting groups permit the accumulation of signal-producing agents in or at specific target structures. Therefore the targeting groups can be selected from such substances, which are principally suitable to provide a purposeful enrichment of the signal generating agents in their specifically available form by physical, chemical or biological routes or combinations thereof. Useful targeting groups to be selected can therefore be antibodies, cell receptor ligands, hormones, lipids, sugars, dextrane, alcohols, bile acids, fatty acids, amino acids, peptides and nucleic acids, which can be chemically or physically attached to signal-generating agents, in order to link the signal-generating agents into/onto a specifically desired structure. In a first embodiment targeting groups are selected, which enrich signal-generating agents in/on a tissue type or on surfaces of cells. Here it is not necessary for the function, that the signal generating agent be taken up into the cytoplasm of the cells. Peptides are preferred as targeting groups, for example chemotactic peptides are used to make inflammation reactions in tissues visible by means of signal generating agents; in this connection see also International Publication No. WO 97/14443.

Antibodies are also preferred, including antibody fragments, Fab, Fab2, Single Chain Antibodies (for example Fv), chimerical antibodies, and the like, as known from the conventional, moreover antibody-like substances, for example so-called anticalines, wherein it is unimportant whether the antibodies are modified after preparation, recombinants are produced or whether they are human or non-human antibodies. It is preferred to choose from humanized or human antibodies, examples of humanized forms of non-human antibodies are chimerical immunoglobulines, immunoglobulin chains or fragments (such as Fv, Fab, Fab′, F(ab″)₂ or other antigen-binding subsequences of antibodies, which partly contain sequences of non-human antibodies; humanized antibodies contain for example human immunoglobulines (receptor or recipient antibody), in which groups of a CDR (Complementary Determining Region) of the receptor are replaced through groups of a CDR of a non-human (spender or donor antibody), wherein the spender species for example, mouse, rabbit or other has appropriate specificity, affinity, and capacity for the binding of target antigens. In a few forms the Fv framework groups of the human immunglobulines are replaced by means of corresponding non-human groups. Humanized antibodies can moreover contain groups which either do not occur in either the CDR or Fv framework sequence of the spender or the recipient. Humanized antibodies essentially comprise substantially at least one or preferably two variable domains, in which all or substantial components of the CDR components of the CDR regions or Fv framework sequences correspond with those of the non-human immunoglobulin, and all or substantial components of the FR regions correspond with a human consensus-sequence. In accordance with the present invention targeting groups of this embodiment can also be hetero-conjugated antibodies. Preferred function of the selected antibodies or peptides are cell surface markers or molecules, particularly of cancer cells, wherein here a large number of known surface structures are known, such as HER2, VEGF, CA15-3, CA 549, CA 27.29, CA 19, CA 50, CA242, MCA, CA125, DE-PAN-2, etc., and the like.

Moreover, it is possible to select targeting groups which contain the functional binding sites of ligands. Such can be chosen from all types, which are suitable for binding to any desired cell receptors. Examples of possible target receptors are, without limiting the choice, receptors of the group of insulin receptors, insulin-like growth factor receptor (e IGF-1 and IGF-2), growth hormone receptor, glucose transporters (particularly GLUT 4 receptor), transferrin receptor (transferrin), Epidermal Growth Factor receptor (EGF), low density lipoprotein receptor, high density lipoprotein receptor, leptin receptor, oestrogen receptor; interleukin receptors including IL-1, IL-2, IL-3, IL-4, IL-5, IL-6, IL-7, IL-8, IL-9, IL-11, IL-12, IL-13, IL-15, and IL-17 receptor, VEGF receptor (VEGF), PDGF receptor (PDGF), Transforming Growth Factor receptor (including TGF-[alpha] and TGF-[beta]), EPO receptor (EPO), TPO receptor (TPO), ciliary neurotrophic factor receptor, prolactin receptor, and T-cell receptors.

It can be preferred to select hormone receptors, especially for hormones, such as steroidal hormones or protein- or peptide-based hormones, for example, however not limited thereto, epinephrines, thyroxines, oxytocine, insulin, thyroid-stimulating hormone, calcitonine, chorionic gonadotropine, corticotropine, follicle stimulating hormone, glucagons, leuteinizing hormone, lipotropine, melanocyte-stimulating hormone, norepinephrines, parathyroid hormone, Thyroid-Stimulating Hormone (TSH), vasopressin's, encephalin, serotonin, estradiol, progesterone, testosterone, cortisone, and glucocorticoide. Receptor ligands include those which are on the cell surface receptors of hormones, lipids, proteins, glycol proteins, signal transducers, growth factors, cytokine, and other bio molecules. Moreover, targeting groups can be selected from carbohydrates with the general formula: Cₓ(H₂O)_{y}, wherein herewith also monosaccharides, disaccharides and oligo—as well as polysaccharides are included, as well as other polymers which consist of sugar molecules which contain glycosidic bonds. Specially preferred carbohydrates are those in which all or parts of the carbohydrate components contain glycosylated proteins, including the monomers and oligomers of galactose, mannose, fructose, galactosamine, glucosamine, glucose, sialic acid, and especially the glycosylated components, which make possible the binding to specific receptors, especially cell surface receptors. Other useful carbohydrates to be selected contain monomers and polymers of glucose, ribose, lactose, raffinose, fructose and other biologically occurring carbohydrates especially polysaccharides, for example, however not exclusively, arabinogalactan, gum Arabica, mannan and the like, which are usable in order to introduce signal generating agents into cells. Reference is made in this connection to U.S. Pat. No. 5,554,386.

Furthermore targeting groups can be selected from the lipid group, wherein also fats, fatty oils, waxes, phospholipids, glycolipids, terpenes, fatty acids and glycerides, especially triglycerides are included. Further included are eicosanoides, steroids, sterols, suitable compounds of which can also be hormones, such as prostaglandins, opiates and cholesterol and the like. In accordance with the present invention, all functional groups can be selected as the targeting group, which possess inhibiting properties, such as for example enzyme inhibitors, preferably those which link signal generating agents into/onto enzymes.

In a further embodiment, targeting groups can be selected from a group of functional compounds which make possible internalization or incorporation of signal generating agents in the cells, especially in the cytoplasm or in specific cell compartments or organelles, such as for example the cell nucleus. For example targeting group is preferred which contains all or parts of HIV-1 tat-proteins, their analogs and derivatized or functionally similar proteins, and in this way allows an especially rapid uptake of substances into the cells. As an example refer to Fawell et al., PNAS USA 91:664 (1994); Frankel et al., Cell 55:1189,(1988); Savion et al., J. Biol. Chem. 256:1149 (1981); Derossi et al., J. Biol. Chem. 269:10444 (1994); and Baldin et al., EMBO J. 9:1511 (1990).

Targeting groups can be further selected from the so-called Nuclear Localisation Signal (NLS), where under short positively charged (basic) domains are understood which bind to specifically targeted structures of cell nuclei. Numerous NLS and their amino acid sequences are known including single basic NLS, such as that of the SV40 (monkey virus) large T Antigen (pro Lys Lys Lys Arg Lys Val), Kalderon (1984), et al., Cell, 39:499-509), the teinoic acid receptor-[beta] nuclear localization signal (ARRRRP); NFKB p50 (EEVQRKRQKL; Ghosh et al., Cell 62:1019 (1990); NFKB p65 (EEKRKRTYE; Nolan et al., Cell 64:961 (1991), as well as others (see for example Boulikas, J. Cell. Biochem. 55(1):32-58 (1994), and double basic NLS's, such as for example xenopus (African clawed toad) proteins, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849, 1988. These are all incorporated herewith by reference in accordance with the present invention. Numerous localization studies have shown that NLSs, which are built into synthetic peptides which normally do not address the cell nucleus or were coupled to reporter proteins, lead to an enrichment of such proteins and peptides in cell nuclei. In this connection exemplary references are made to Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990. It can be especially preferred to select targeting groups for the hepatobiliary system, wherein in U.S. Pat. Nos. 5,573,752 and 5,582,814 corresponding groups are suggested.

In certain exemplary embodiments, the implant comprises absorptive agents, e.g., to remove compounds from body fluids. Suitable absorptive agents, but not exclusively and not limited to, are chelating agents, such as penicillamine, methylene tetramine dihydrochloride, EDTA, DMSA or deferoxamine mesylate, any other appropriate chemical modification of the coating surface, antibodies, and microbeads or other materials containing cross linked reagents for absorption of drugs, toxins or other agents.

In further exemplary embodiments, biologically active agents are selected from cells, cell cultures, organized cell cultures, tissues, organs of desired species and non-human organisms.

In certain exemplary embodiments, the beneficial agents comprise metal based nano-particles that are selected from ferromagnetic or superparamagnetic metals or metal-alloys, either further modified by coating with silanes or any other suitable polymer or not modified, for interstitial hyperthermia or thermoablation. In further exemplary embodiments, the exemplary implants can comprise silver nano-particles or other anti-infective inorganic materials, preferably as nano-particles with a D50 between about 10 nm and 50 nm, whereby the amount of the anti-infective particles is at least about 1 weight %, preferably about 2-5 weight % and more preferably about 5 to 10 weight %, even more preferably between 10 and 20 weight %.

In another exemplary embodiment, it can be desirable to coat the implant on the outer surface or inner surface with a coating to enhance engraftment or biocompatibility. Such coatings may comprise carbon coatings, metal carbides, metal nitrides, metal oxides e.g., diamond-like carbon or silicon carbide, or pure metal layers of e.g., titanium, using PVD, Sputter-, CVD or similar vapor deposition methods or ion implantation.

In further exemplary embodiments, it can be preferred to produce a porous coating onto at least one part of the exemplary implant in a further step, such as porous carbon coatings as described in International Publication Nos. WO 2004/101177, WO 2004/101017 or WO 2004/105826, or porous composite-coatings as described in International Application No. PCT/EP2006/063450, or porous metal-based coatings as described in International Publication No. WO2006/097503, or any other suitable porous coating.

In further exemplary embodiments, a sol/gel-based beneficial agent can be incorporated into the exemplary implant or a sol/gel-based coating that can be dissolvable in physiologic fluids may be applied to at least a part of the implant, as described in, e.g., International Publication Nos. WO 2006/077256 or WO 2006/082221.

In some exemplary embodiments, it can be desirable to combine two or more different functional modifications as described above to obtain a functional implant.

It should be noted that the term ‘comprising’ does not exclude other elements or steps and the ‘a’ or ‘an’ does not exclude a plurality. In addition elements described in association with the different embodiments may be combined.

It should be noted that the reference signs in the claims shall not be construed as limiting the scope of the claims.

Having thus described in detail several exemplary embodiments of the present invention, it is to be understood that the present invention described above is not to be limited to particular details set forth in the above description, as many apparent variations thereof are possible without departing from the spirit or scope of the present invention. The exemplary embodiments of the present invention are disclosed herein or are obvious from and encompassed by the detailed description. The detailed description, given by way of example, but not intended to limit the present invention solely to the specific embodiments described, may best be understood in conjunction with the accompanying Figures.

The foregoing applications, and all documents cited therein or during their prosecution (“appln. cited documents”) and all documents cited or referenced in the appln. cited documents, and all documents cited or referenced herein (“herein cited documents”), and all documents cited or referenced in the herein cited documents, together with any manufacturer's instructions, descriptions, product specifications, and product sheets for any products mentioned herein or in any document incorporated by reference herein, are hereby incorporated herein by reference, and may be employed in the practice of the present invention.

What is claimed is:

1. An at least partially biodegradable implant suitable for implantation into a subject for repairing a bone or cartilage defect, comprising:
a matrix of a non-particulate material, the matrix including an open-celled structure having a plurality of interconnected spaces, and
a plurality of particles of a metallic material,
wherein the interconnected spaces in the matrix are substantially completely filled with the metallic material particles, and wherein at least one of the metallic material or the non-particulate material is at least partially degradable in-vivo.

2. The implant of claim 1, wherein the matrix has a bulk volume porosity of about 10-90%.

3. The implant of claim 2, wherein the matrix has a spongy or trabecular open-celled lattice structure, and wherein the interconnected spaces are formed by at least one of interconnected pores, channels or pores.

4. The implant of claim 1, wherein the at least one of the spaces, channels or pores have a dimension suitable for osteoconduction of about 200 to 1000 μm.

5. The implant of claim 1, wherein the metallic material includes at least one of a metal or a metal alloy.

6. The implant of claim 1, wherein the metallic material particles are completely degradable in-vivo.

7. The implant of claim 6, wherein the metallic material is one of a metal or an alloy.

8. The implant of claim 6, wherein the metallic material includes at least one metal selected from an alkaline metal, an alkaline earth metal, Fe, Zn, Al, Mg, Ca, Zn, W, Ln, Si, or Y.

9. The implant of claim 6, wherein the degradable metallic material is combined with other metallic particles selected from at least one of Mn, Co, Ni, Cr, Cu, Cd, Pb, Sn, Th, Zr, Ag, Au, Pd, Pt, Si, Ca, Li, Al, Zn or Fe.

10. The implant of claim 6, wherein the degradable metallic material includes a magnesium alloy comprising more than about 90% of Mg, about 4-5% of Y, and about 1.5-4% of other rare earth metals.

11. The implant of claim 6, wherein the degradable metallic material particles comprises a metal alloy of one of:
(i) about 10-98 wt.-% of Mg, and about 0-70 wt.-% of Li and 0-12 wt.-% of other metals,
(ii) about 60-99 wt.-% of Fe, about 0.05-6 wt.-% Cr, about 0.05-7 wt.-% Ni and up to about 10 wt.-% of other metals; or
(iii) about 60-96 wt.-% Fe, about 1-10 wt.-% Cr, about 0.05-3 wt.-% Ni and about 0-15 wt.-% of other metals,
wherein individual weight ranges are selected to add up to about 100 wt.-% in total for each alloy.

12. The implant of claim 5, wherein the metallic material particles are substantially not degradable in-vivo.

13. The implant of claim 12, wherein the metallic material includes at least one metal selected from the group of main group metals of the periodic system, transition metals, such as copper, gold and silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum, or from rare earth metals.

14. The implant of claim 5, wherein the metallic material includes a biocorrosive alloy, such as biocorrosive alloys comprising as a major component tungsten, rhenium, osmium or molybdenum.

15. The implant of claim 14, wherein the biocorrosive alloy further comprises cerium, an actinide, iron, tantalum, platinum, gold, gadolinium, yttrium or scandium.

16. The implant of claim 5, wherein the metallic material particles comprise a mixture of at least one first metallic material and at least one second metallic material, the first metallic material being more electronegative than the second metallic material, such that the first and second metallic material particles form a local cell at their contact surfaces.

17. The implant of claim 1, wherein the average particle size (D50) of the metallic material is from about 0.5 nm to about 5000 μm.

18. The implant of claim 1, wherein the non-particulate matrix material is an organic material.

19. The implant of claim 18, wherein the organic material comprises an oligomer, polymer or copolymer including at least one of a poly(meth)acrylate, unsaturated polyester, saturated polyester, polyolefines, polyethylene, polypropylene, polybutylene, alkyd resins, epoxy-polymers or resins, polyamide, polyimide, polyetherimide, polyamideimide, polyesterimide, polyester amide imide, polyurethane, polycarboxylate, polycarbonate, polystyrene, polyphenol, polyvinyl ester, polysilicone, polyacetal, cellulosic acetate, polyvinylchloride, polyvinyl acetate, polyvinyl alcohol, polysulfone, polyphenylsulfone, polyethersulfone, polyketone, polyetherketone, polybenzimidazole, polybenzoxazole, polybenzthiazole, polyfluorocarbons, polyphenylene ether, polyarylate, or cyanatoester-polymers, and any of the copolymers and any mixtures thereof.

20. The implant of claim 18, wherein the organic material comprises a polymer or copolymer selected from at least one of collagen, albumin, gelatin, hyaluronic acid, starch, cellulose, methylcellulose, hydroxypropylcellulose, hydroxypropylmethyl-cellulose, carboxymethylcellulose-phtalate; gelatin, casein, dextrane, polysaccharide, fibrinogen, poly(D,L lactide), poly(D,L-lactide-co-glycolide), poly(glycolide-co-trimethylene carbonates), poly(glycolide), poly(hydroxybutylate), poly(alkylcarbonate), poly(a-hydroxyesters), poly(ether esters, poly(orthoester), polyester, poly(hydroxyvaleric acid), polydioxanone, poly(ethylene terephtalate), poly(maleic acid), poly(malic acid), poly(tartaric acid), polyanhydride, polyphosphazene, poly(amino acids), polypeptides, polycaprolactones, poly(propylene fumarates), poly(ester amides), poly(ethylene fumarates), poly(hydroxy butyrates), and polyurethanes.

21. The implant of claim 18, wherein the organic material is at least partially biodegradable in-vivo.

22. The implant of claim 1, wherein the non-particulate matrix material includes an inorganic-organic hybrid material, obtainable by sol-gel processing.

23. The implant of claim 1, wherein the non-particulate matrix material includes a metal, a metal alloy or a ceramic material.

24. The implant of claim 1, further comprising at least one additive including an inorganic or organic filler.

25. The implant of claim 24, wherein the beneficial agent includes at least one of a pharmacologically, therapeutically, biologically or diagnostically active agent or an absorptive agent.

26. The implant of claim 25, wherein the beneficial ingredient is configured to be released in-vivo from the final implant.

27. The implant of claim 1, wherein the particles of metallic material comprise at least about 5 wt of the implant.

28. The implant of claim 1, wherein the matrix material comprises at least about 5 wt.-% of the implant.

29. The implant of claim 1, wherein the implant has a Youngs modulus corresponding to cancellous natural bone in the range from about 0.01 to about 2 GPa.

30. The implant of claim 1, wherein the implant has a Youngs modulus corresponding to cortical natural bone in the range from about 15 to about 30 GPa.

31. The implant of claim 1, wherein the matrix material is substantially non-degradable in-vivo.

32. The implant of claim 1, wherein the matrix material and the metallic material particles are degradable in-vivo.

33. The implant of claim 32, wherein the in-vivo degradation rate of the matrix material and the metallic material particles are different.

34. The implant of claim 33, wherein the in-vivo degradation rate of the matrix material is lower than the degradation rate of the metallic material particles.

35. The implant of claim 33, wherein the in-vivo degradation rate of the matrix material is higher than the degradation rate of the metallic material particles.

36. The implant of claim 31, wherein the metallic material particles are selected such that the in-vivo degradation rate of the particles matches with the re-growth or repair rate of the natural bone, and wherein the degradation rate of the particles is in a range of from about 3 to 8 weeks.

37. The implant of claim 31, wherein the metallic material particles are selected such that the in-vivo degradation rate of the particles matches with the regrowth or repair rate of the natural cartilage, and wherein the degradation rate of the particles is in a range of from about 4 to 10 weeks.

38. The implant of claim 1, wherein the implant is selected from one of a tissue or cartilage scaffold, an implantable fracture fixation device, such as plates, screws and rods, a dental implant, an orthopedic implant, a traumatologic implant, or a surgical implant.

39. A method for repairing a bone or cartilage defect in a living organism, comprising implanting an implant into the defective bone or cartilage or replacing the defective bone or cartilage at least partially, wherein the implant is an at least partially biodegradable implant suitable for implantation into a subject for repairing a bone or cartilage defect, the implant comprising:
a matrix of a non-particulate material, the matrix including an open-celled structure having a plurality of interconnected spaces, and
a plurality of particles of a metallic material,
wherein the interconnected spaces in the matrix are substantially completely filled with the metallic material particles, and wherein at least one of the metallic material or the non-particulate material is at least partially degradable in-vivo.

40. The method of claim 39, wherein the defect includes a defect or a wound in a bone, a tooth or a cartilage of a living organism.

41. A utilization of an implant, the implant being an at least partially biodegradable implant suitable for implantation into a subject for repairing a bone or cartilage defect, the implant comprising:
a matrix of a non-particulate material, the matrix including an open-celled structure having a plurality of interconnected spaces, and
a plurality of particles of a metallic material,
wherein the interconnected spaces in the matrix are substantially completely filled with the metallic material particles, and wherein at least one of the metallic material or the non-particulate material is at least partially degradable in-vivo
wherein for repairing a bone, tooth or cartilage defect in a living organism.
